(19) United States
(12) Patent Application Publication
(10) Pub. No.: US 2026/0250403 A1
(43) Pub. Date: Aug. 27, 2026

(54) ANTI-CD40 ANTIBODIES AND USES THEREOF

(71) Applicant: Biocytogen Pharmaceuticals (Beijing) Co., Ltd., Beijing (CN)

(72) Inventors: Yacui Liu, Beijing (CN); Maopeng Tian, Beijing (CN); Fang Yang, Beijing (CN); Yi Yang, Beijing (CN)

(21) Appl. No.: 18/855,369

(22) PCT Filed: Apr. 14, 2023
(86) PCT No.: PCT/CN2023/088417

§ 371 Date: Oct. 9, 2024

(30) Foreign Application Priority Data
Apr. 15, 2022 (WO) PCT/CN2022/087003

(51) Int. Cl. C07K 16/28; A01K 67/0278; A61K 39/00; A61K 47/68; A61P 19/02; A61P 25/28; A61P 37/04

(52) U.S. Cl. CPC C07K 16/2878; A01K 67/0278; A61K 47/6849; A61P 19/02; A61P 25/28; A61P 37/04; A01K 2227/105; A61K 2039/505; C07K 2317/14; C07K 2317/31; C07K 2317/33; C07K 2317/52; C07K 2317/565; C07K 2317/622; C07K 2317/71; C07K 2317/732; C07K 2317/92; C07K 2317/94

(57) ABSTRACT

This disclosure relates to anti-CD40 (TNF Receptor Superfamily Member 5) antibodies, antigen-binding fragments, and the uses thereof.

### CLAIM OF PRIORITY

This application claims the benefit of PCT application App. No. PCT/CN2022/087003, filed on Apr. 15, 2022. The entire contents of the foregoing application are incorporated herein by reference.

### TECHNICAL FIELD

This disclosure relates to anti-CD40 (TNF Receptor Superfamily Member 5) antibodies and uses thereof.

### BACKGROUND

Autoimmune diseases are conditions arising from an abnormal immune response to a normal body part. There are at least 80 types of autoimmune diseases. The cause of autoimmune disease is generally not well understood. Some autoimmune diseases such as lupus run in families, and some other autoimmune diseases may be triggered by infections or other environmental factors. Some common autoimmune diseases include e.g., celiac disease, diabetes mellitus type 1, Graves' disease, inflammatory bowel disease, multiple sclerosis, psoriasis, rheumatoid arthritis, and systemic lupus erythematosus.

Recent clinical and commercial success of therapeutic antibodies has created great interest in using antibodies to treat various immune-related disorders. There is a need to develop antibodies for use in various antibody-based therapeutics to treat autoimmune diseases.

### SUMMARY

This disclosure relates to anti-CD40 antibodies, antigen-binding fragment thereof, and the uses thereof.

In one aspect, the disclosure is related to an antibody or antigen-binding fragment thereof that binds to CD40 (TNF Receptor Superfamily Member 5) comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(4) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(5) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 28, 29, 30, respectively; and
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively.

In some embodiments, the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 4, 5, and 6, respectively. In some embodiments, the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively. In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively. In some embodiments, the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 22, 23, and 24, respectively. In some embodiments, the VH comprises CDRs 1, 2, 3 with the amino acid sequences set forth in SEQ ID NOs: 25, 26 and 27, respectively, and the VL comprises CDRs 1, 2, 3 with the amino acid sequences set forth in SEQ ID NOs: 28, 29, and 30, respectively. In some embodiments, the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 34, 35, and 36, respectively.

In some embodiments, the antibody or antigen-binding fragment thereof specifically binds to human, monkey, or dog CD40. In some embodiments, the antibody or antigen-binding fragment thereof is a human antibody or antigen-binding fragment thereof. In some embodiments, the antibody or antigen-binding fragment thereof is a single-chain variable fragment (scFV) or a multi-specific antibody (e.g., a bispecific antibody). In some embodiments, the antibody or antigen-binding fragment thereof is a human IgG1 antibody or antigen-binding fragment thereof (optionally with YTE and/or LALA mutations) or a human IgG4 antibody or antigen-binding fragment thereof (optionally with YTE).

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and in some embodiments, the VH, when paired with a light chain variable region (VL) comprising the amino acid sequence set forth in SEQ ID NO: 37 binds to CD40;
(2) an immunoglobulin light chain or a fragment thereof comprising a VL comprising complementarity determining regions (CDRs) 1, 2, and 3 comprising the amino acid sequences set forth in SEQ ID NOs: 4, 5, and 6, respectively, and in some embodiments, the VL, when paired with a VH comprising the amino acid sequence set forth in SEQ ID NO: 65 binds to CD40;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and in some embodiments, the VH, when paired with a light chain variable region (VL) comprising the amino acid sequence set forth in SEQ ID NO: 39 binds to CD40;
(4) an immunoglobulin light chain or a fragment thereof comprising a VL comprising complementarity determining regions (CDRs) 1, 2, and 3 comprising the amino acid sequences set forth in SEQ ID NOs: 10, 11, and 12, respectively, and in some embodiments, the VL, when paired with a VH comprising the amino acid sequence set forth in SEQ ID NO: 38 binds to CD40;
(5) an immunoglobulin heavy chain or a fragment thereof comprising a VH comprising complementarity determining regions (CDRs) 1, 2, and 3 comprising the amino acid sequences set forth in SEQ ID NOs: 13, 14, and 15, respectively, and in some embodiments, the VH, when paired with a light chain variable region (VL) comprising the amino acid sequence set forth in SEQ ID NO: 41 binds to CD40; or
(6) an immunoglobulin light chain or a fragment thereof comprising a VL comprising CDRs 1, 2, and 3 comprising the amino acid sequences set forth in SEQ ID NOs: 16, 17 and 18, respectively, and in some embodiments, the VL, when paired with a VH comprising the amino acid sequence set forth in SEQ ID NO: 40 binds to CD40.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

In some embodiments, the VH when paired with a VL specifically binds to human, monkey, or dog CD40, or the VL when paired with a VH specifically binds to human, monkey, or dog CD40. In some embodiments, the immunoglobulin heavy chain or the fragment thereof is a human immunoglobulin heavy chain or a fragment thereof (e.g., a human IgG1 heavy chain or a fragment thereof, optionally with YTE and/or LALA mutations; or a human IgG4 heavy chain or a fragment thereof, optionally with YTE), and the immunoglobulin light chain or the fragment thereof is a human immunoglobulin light chain or a fragment thereof. In some embodiments, the nucleic acid encodes a single-chain variable fragment (scFv) or a multi-specific antibody (e.g., a bispecific antibody). In some embodiments, the nucleic acid is cDNA.

In one aspect, the disclosure is related to a vector comprising one or more of the nucleic acids as described herein. In one aspect, the disclosure is related to a vector comprising two of the nucleic acids as described herein, in some embodiments, the vector encodes the VL region and the VH region that together bind to CD40. In one aspect, the disclosure is related to a pair of vectors, in some embodiments, each vector comprises one of the nucleic acids as described herein, in some embodiments, together the pair of vectors encodes the VL region and the VH region that together bind to CD40.

In one aspect, the disclosure is related to a cell comprising the vector, or the pair of vectors as described herein. In some embodiments, the cell is a CHO cell. In one aspect, the disclosure is related to a cell comprising one or more of the nucleic acids as described herein. In one aspect, the disclosure is related to a cell comprising two of the nucleic acids as described herein. In some embodiments, the two nucleic acids together encode the VL region and the VH region that together bind to CD40.

In one aspect, the disclosure is related to a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

In one aspect, the disclosure is related to an antibody or antigen-binding fragment thereof that binds to CD40 comprising a heavy chain variable region (VH) comprising an amino acid sequence that is at least 90% identical to a selected VH sequence, and a light chain variable region (VL) comprising an amino acid sequence that is at least 90% identical to a selected VL sequence, in some embodiments, the selected VH sequence and the selected VL sequence are one of the following: (1) the selected VH sequence is SEQ ID NO: 65, and the selected VL sequence is SEQ ID NO: 37; (2) the selected VH sequence is SEQ ID NO: 38, and the selected VL sequence is SEQ ID NO: 39; and (3) the selected VH sequence is SEQ ID NO: 40, and the selected VL sequence is SEQ ID NO: 41.

In some embodiments, the VH comprises the sequence of SEQ ID NO: 65 and the VL comprises the sequence of SEQ ID NO: 37. In some embodiments, the VH comprises the sequence of SEQ ID NO: 38 and the VL comprises the sequence of SEQ ID NO: 39. In some embodiments, the VH comprises the sequence of SEQ ID NO: 40 and the VL comprises the sequence of SEQ ID NO: 41.

In some embodiments, the antibody or antigen-binding fragment thereof specifically binds to human, monkey, or dog CD40. In some embodiments, the antibody or antigen-binding fragment thereof is a human antibody or antigen-binding fragment thereof. In some embodiments, the antibody or antigen-binding fragment is a single-chain variable fragment (scFV) or a multi-specific antibody (e.g., a bispecific antibody). In some embodiments, the antibody or antigen-binding fragment is a human IgG1 antibody or antigen-binding fragment thereof (optionally with YTE and/or LALA mutations) or a human IgG4 antibody or antigen-binding fragment thereof (optionally with YTE).

In one aspect, the disclosure is related to an antibody or antigen-binding fragment thereof comprising the VH CDRs 1, 2, 3, and the VL CDRs 1, 2, 3 of the antibody or antigen-binding fragment thereof as described herein.

In one aspect, the disclosure is related to an antibody or antigen-binding fragment thereof that cross-competes with the antibody or antigen-binding fragment thereof as described herein.

In one aspect, the disclosure is related to an antibody-drug conjugate comprising the antibody or antigen-binding fragment thereof as described herein covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof, or the antibody-drug conjugate as described herein, to the subject. In some embodiments, the subject has a solid tumor. In some embodiments, the cancer is melanoma, pancreatic carcinoma, mesothelioma, or a hematological malignancy. In some embodiments, the cancer is Non-Hodgkin's lymphoma, lymphoma, or chronic lymphocytic leukemia.

In one aspect, the disclosure is related to a method of decreasing the rate of tumor growth, the method comprising contacting a tumor cell with an effective amount of a composition comprising an antibody or antigen-binding fragment thereof, or the antibody-drug conjugate as described herein. In one aspect, the disclosure is related to a method of killing a tumor cell, the method comprising contacting a tumor cell with an effective amount of a composition comprising the antibody or antigen-binding fragment thereof, or the antibody-drug conjugate as described herein. In one aspect, the disclosure is related to a method of inhibiting immune response in a subject, the method comprising administering to the subject an effective amount of a composition comprising the antibody or antigen-binding fragment thereof, or the antibody-drug conjugate as described herein. In some embodiments, the subject has an autoimmune disease.

In one aspect, the disclosure is related to a method of treating an autoimmune disease, the method comprising administering to the subject an effective amount of a composition comprising the antibody or antigen-binding fragment thereof, or the antibody-drug conjugate as described herein. In some embodiments, the autoimmune disease is rheumatoid arthritis, systemic lupus erythematosus, lupus nephritis, allergic dermatitis, or multiple sclerosis.

In one aspect, the disclosure is related to a pharmaceutical composition comprising the antibody or antigen-binding fragment thereof as described herein, and a pharmaceutically acceptable carrier. In one aspect, the disclosure is related to a pharmaceutical composition comprising the antibody drug conjugate as described herein, and a pharmaceutically acceptable carrier.

In one aspect, the disclosure is related to an antibody or antigen-binding fragment thereof that binds to CD40 comprising a Fc region, in some embodiments, the Fc region lacks ADCC effect or has reduced ADCC effect as compared to a wild-type Fc region. In some embodiments, the KD between the antibody or antigen-binding fragment thereof and FcRn (e.g., human FcRn) is less than 1×10⁻⁵ M, less than 5×10⁻⁶ M, less than 1×10⁻⁶ M, less than 5×10⁻⁷ M, less than 1×10⁻⁷ M, or less than 5×10⁻⁸ M. In some embodiments, the Fc region is IgG1 or IgG4 subtype. In some embodiments, the Fc region comprises YTE mutations. In some embodiments, the Fc region comprises LALA mutations. In some embodiments, the half-life of the antibody or antigen-binding fragment thereof as described herein is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days, when administered to a subject. In some embodiments, the subject is a mouse. In some embodiments, the subject is genetically-modified to express a human or humanized CD40.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains. In some embodiments, the multimeric antibody is able to crosslink one target molecule (e.g., CD40) to at least one second target molecule (e.g., CTLA-4) on the surface of a mammalian cell (e.g., a human T-cell).

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., CD40) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a CD40 molecule may be referred to as a CD40-specific antibody or an anti-CD40 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows experimental scheme to analyze the effects of anti-CD40 antibodies on immune responses in hCD40 mice. Anti-CD40 antibodies were administered on Day 0 and Day 4. OVA and CFA were administered on Day 1. Anti-OVA antibody level was determined by ELISA on Day 10 and Day 17.

FIG. 2 shows the anti-OVA antibody level on Day 10 determined by ELISA. The hCD40 mice were administered with PBS (G1), Bleselumab analog (G2), 12B5-IgG4-FLAA (G3), 16D5-IgG4-FLAA (G4), 2F8-IgG4-FLAA (G5), 6A4-IgG4-FLAA (G6), or 15B4-IgG4-FLAA (G7).

FIG. 3 shows the anti-OVA antibody level on Day 17 determined by ELISA.

FIG. 4 shows concentrations of Bleselumab analog (G1), 12B5-IgG4-FLAA (G2), 6A4-IgG4-FLAA (G3), and 2F8-IgG4-FLAA (G4) in the serum of hCD40 mice over time and concentrations of Bleselumab analog (G5), 12B5-IgG4-FLAA (G6), 6A4-IgG4-FLAA (G7), and 2F8-IgG4-FLAA (G8) in the serum of C57BL/6 mice over time.

FIG. 5 shows concentrations of 12B5-IgG1-LALA-YTE (G1), 12B5-IgG1-N297A-YTE (G2), 12B5-IgG4-YTE (G3), and 12B5-IgG4-FLAA (G4) in the serum of hFcRn mice over time.

FIG. 6 shows the anti-OVA antibody level on Day 10 determined by ELISA. The hCD40/hFcRn mice were administered with PBS (G1), Bleselumab analog (G2-G4), 12B5-IgG1-LALA-YTE (G5-G7), or 2F8-IgG1-LALA-YTE (G8-G10). The anti-CD40 antibodies were administered on Day 0. OVA and CFA were administered on Day 1.

FIG. 7 shows the anti-OVA antibody level on Day 17 determined by ELISA.

FIG. 8 shows the anti-OVA antibody level on Day 21 determined by ELISA.

FIG. 9 lists CDR sequences of anti-CD40 antibodies (2F8, 6A4, and 12B5) and CDR sequences of related anti-CD40 antibodies thereof as defined by Kabat numbering.

FIG. 10 lists CDR sequences of anti-CD40 antibodies (2F8, 6A4, and 12B5) and CDR sequences of related anti-CD40 antibodies thereof as defined by Chothia numbering.

FIG. 11 lists amino acid sequences of heavy chain variable regions and light chain variable regions of anti-CD40 antibodies (2F8, 6A4, and 12B5).

FIG. 12 shows cytotoxicity data for 12B5-IgG1-LALA-YTE, 12B5-IgG1, Rituximab analog and human IgG1.

FIG. 13 shows cytotoxicity data for 12B5-IgG1-LALA-YTE, Rituximab analog and human IgG1.

FIG. 14 shows the inhibitory effect of 12B5-IgG1-LALA-YTE and human IgG1 on PBMC proliferation.

FIG. 15A shows the inhibitory effect of 12B5-IgG1-LALA-YTE on B cells proliferation.

FIG. 15B shows the inhibitory effect of hIgG1 on B cells proliferation.

FIG. 16 shows the body weight of hCD40/hFcRn mice that were injected with MOG to induce an EAE model, and then treated with hIgG1 (G2) or anti-hCD40 antibody 2F8-IgG1-LALA-YTE (G3). hCD40/hFcRn mice injected with PBS were used as control (G1).

FIG. 17 shows the clinical score of hCD40/hFcRn mice that were injected with MOG to induce an EAE model, and then treated with hIgG1 (G2) or anti-hCD40 antibody 2F8-IgG1-LALA-YTE (G3). hCD40/hFcRn mice injected with PBS were used as control (G1).

FIG. 18 shows H&E (hematoxylin and eosin) staining images of spinal cords from hCD40/hFcRn mice that were injected with MOG to induce an EAE model, and then treated with hIgG1 (G2) or anti-hCD40 antibody 2F8-IgG1-LALA-YTE (G3). hCD40/hFcRn mice injected with PBS were used as control (G1). The images were examined for inflammatory cell infiltration.

FIG. 19 shows LFB (Luxol Fast Blue) staining images of spinal cords from hCD40/hFcRn mice that were injected with MOG to induce an EAE model, and then treated with hIgG1 (G2) or anti-hCD40 antibody 2F8-IgG1-LALA-YTE (G3). hCD40/hFcRn mice injected with PBS were used as control (G1). The images were examined for spinal cord demyelination.

FIG. 20 shows the body weight of hCD40 mice that were injected with CII emulsion to induce a CIA model, and then treated with PBS (G2) or anti-hCD40 antibody 12B5-IgG1-LALA-YTE (G3). hCD40 mice injected with PBS only were used as control (G1).

FIG. 21 shows the clinical score of hCD40 mice that were injected with CII emulsion to induce a CIA model, and then treated with PBS (G2) or anti-hCD40 antibody 12B5-IgG1-LALA-YTE (G3). hCD40 mice injected with PBS only were used as control (G1).

FIG. 22 shows the incidence rate of hCD40 mice that were injected with CII emulsion to induce a CIA model, and then treated with PBS (G2) or anti-hCD40 antibody 12B5-IgG1-LALA-YTE (G3). hCD40 mice injected with PBS only were used as control (G1).

FIG. 23 shows H&E staining images of joint tissues collected from the four limbs of hCD40 mice that were injected with CII emulsion to induce a CIA model, and then treated with PBS (G2) or anti-hCD40 antibody 12B5-IgG1-LALA-YTE (G3). hCD40 mice injected with PBS only were used as control (G1). (a) stands for inflammatory cell infiltration, (b) stands for synovial hyperplasia, and (c) stands for pannus.

FIG. 24 lists amino acid sequences discussed in the disclosure.

### DETAILED DESCRIPTION

The present disclosure provides examples of antibodies, antigen-binding fragment thereof, that bind to CD40 (TNF Receptor Superfamily Member 5).

### CD40 and Immune System

The immune system can differentiate between normal cells in the body and those it sees as “foreign,” which allows the immune system to attack the foreign cells while leaving the normal cells alone. This mechanism sometimes involves proteins called immune checkpoints. Immune checkpoints are molecules in the immune system that either turn up a signal (co-stimulatory molecules) or turn down a signal.

Checkpoint inhibitors can prevent the immune system from attacking normal tissue and thereby preventing autoimmune diseases. Many tumor cells also express checkpoint inhibitors. These tumor cells escape immune surveillance by co-opting certain immune-checkpoint pathways, particularly in T cells that are specific for tumor antigens (Creelan, Benjamin C. “Update on immune checkpoint inhibitors in lung cancer.” Cancer Control 21.1 (2014): 80-89). Because many immune checkpoints are initiated by ligand-receptor interactions, they can be readily blocked by antibodies against the ligands and/or their receptors.

CD40 (also known as Tumor Necrosis Factor Receptor Superfamily Member 5 or TNFRSF5) is a tumor necrosis factor receptor superfamily member expressed on antigen presenting cells (APC) such as dendritic cells (DC), macrophages, B cells, and monocytes as well as many non-immune cells and a wide range of tumors. Interaction with its trimeric ligand CD154 (also known as CD40 ligand or CD40L) on activated T helper cells results in APC activation, leading to the induction of adaptive immunity.

Physiologically, signaling via CD40 on APC is thought to represent a major component of T cell help and mediates in large part the capacity of helper T cells to license APC. Ligation of CD40 on DC, for example, induces increased surface expression of costimulatory and MHC molecules, production of proinflammatory cytokines, and enhanced T cell triggering. CD40 ligation on resting B cells increases antigen-presenting function and proliferation.

In pre-clinical models, rat anti-mouse CD40 mAb show remarkable therapeutic activity in the treatment of CD40+ B-cell lymphomas (with 80-100% of mice cured and immune to re-challenge in a CD8 T-cell dependent manner) and are also effective in various CD40-negative tumors. These mAb are able to clear bulk tumors from mice with near terminal disease. CD40 mAb have been investigated in clinical trials and are used for treating melanoma, pancreatic carcinoma, mesothelioma, hematological malignancies, especially Non-Hodgkin's lymphoma, lymphoma, chronic lymphocytic leukemia, and advanced solid tumors.

Therapeutic anti-CD40 antibodies show diverse activities ranging from strong agonism to antagonism. Currently there is no satisfactory explanation for this heterogeneity. The primary mechanistic rationale invoked for agonistic CD40 mAb is to activate host APC in order to induce clinically meaningful anti-tumor T-cell responses in patients. These include T cell-independent but macrophage-dependent triggering of tumor regression. CD40-activated macrophages can become tumoricidal, and least in pancreatic cancer, may also facilitate the depletion of tumor stroma which induces tumor collapse in vivo. Importantly, these mechanisms do not require expression of CD40 by the tumor, which has justified inclusion of patients with a broad range of tumors in many of the clinical trials. Insofar as these strategies aim to activate DC, macrophages, or both, the goal is not necessarily for the CD40 mAb to kill the cell it binds to, for example, via complement mediated cytotoxicity (CMC) or antibody dependent cellular cytoxicity (ADCC). Thus, by design, the strong agonistic antibody does not mediate CMC or ADCC.

In contrast, other human CD40 mAb can mediate CMC and ADCC against CD40+ tumors, such as nearly all B cell malignancies, a fraction of melanomas, and certain carcinomas. Finally, there is some evidence that ligation of CD40 on tumor cells promotes apoptosis and that this can be accomplished without engaging any immune effector pathway. This has been shown for CD40+ B cell malignancies and certain solid tumors such as CD40+ carcinomas and melanomas.

Because of the centrality of CD40 in generating effective immune responses, CD40 also plays an important role in the pathogenesis of autoimmune disease. CD40 contributes to T-cell dependent autoimmune diseases in several ways. First, CD40 signaling can function at the level of T cell selection in the thymus. Medullary thymic epithelial cells (mTECs) mediate negative selection of potentially autoreactive T cells by expressing peripheral tissue-restricted antigens. While the TNFR family member RANK is critically important in embryonic mTEC development, CD40 cooperates with RANK in promoting mTEC development after birth and thus self-tolerance. Disruption of CD40-CD154 interactions in mTECs could potentially contribute to failure of central tolerance. Secondly, CD40 signaling results in the production of pro-inflammatory cytokines, such as IL-6, which can influence T cell differentiation to Th17 cells. CD40 is also upregulated upon antigen presenting cell (APC) activation. Increased levels of CD40, either constitutive or induced, can contribute to increased strength of CD40-CD154 interactions. Another mechanism can be aberrant expression of CD40 in tissues where it is normally undetectable. It has been hypothesized that aberrant expression of MHC class II molecules on endocrine tissues could contribute to the initiation of autoimmune disease. Aberrant CD40 expression on such tissues has also been proposed as a contributing factor to the initiation of autoimmunity in Grave's disease, and in the production of inflammatory cytokines contributing to the failure of pancreatic islet cell transplants. Finally, CD40 bearing CD4+ T cells play a role in type 1 diabetes in humans and mice. Thus, CD40 is an attractive candidate receptor for contributing to a variety of autoimmune processes in which B and T cell activation play a role in pathogenesis.

A detailed description of CD40 and its function can be found, e.g., in Vonderheide et al., “Agonistic CD40 antibodies and cancer therapy.” (2013): 1035-1043; Beatty, et al. “CD40 agonists alter tumor stroma and show efficacy against pancreatic carcinoma in mice and humans.” Science 331.6024 (2011): 1612-1616; Vonderheide, et al. “Clinical activity and immune modulation in cancer patients treated with CP-870,893, a novel CD40 agonist monoclonal antibody.” Journal of Clinical Oncology 25.7 (2007): 876-883; Peters et al., “CD40 and autoimmunity: the dark side of a great activator.” Seminars in immunology. Vol. 21. No. 5. Academic Press, 2009; each of which is incorporated by reference in its entirety.

The present disclosure provides several anti-CD40 antibodies, antigen-binding fragments thereof, and methods of using these anti-CD40 antibodies and antigen-binding fragments to inhibit tumor growth and to treat cancers.

### Antibodies and Antigen Binding Fragments

The present disclosure provides anti-CD40 antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety. Unless specifically indicated in the present disclosure, Kabat numbering is used in the present disclosure as a default.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV has one heavy chain variable domain, and one light chain variable domain. In some embodiments, the scFV has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting CD40 and an addition antigen (e.g., TNFRSF9 (4-1BB), MSLN, FAP, ALB, CTLA4, HER2, GPC3, MYC, EPCAM, TNFRSF14 (LIGHTR), or ITGAX (CD11c).

### Anti-CD40 Antibodies and Antigen-Binding Fragments

The disclosure provides antibodies and antigen-binding fragments thereof that specifically bind to CD40 (e.g., human CD40). The antibodies and antigen-binding fragments described herein are capable of binding to CD40. These antibodies can be agonists or antagonists. In some embodiments, these antibodies can promote CD40 signaling pathway thus increase immune response. In some embodiments, the antibodies can block CD40 signaling pathway thus reduce immune response. In some embodiments, these antibodies can initiate complement-dependent cytotoxicity (CMC) or antibody-dependent cellular cytotoxicity (ADCC).

The disclosure provides e.g., mouse anti-CD40 antibodies 2F8, 6A4, 12B5, the chimeric antibodies thereof, and the human or humanized antibodies thereof.

The CDR sequences for 2F8, and 2F8 derived antibodies (e.g., human or humanized antibodies) include CDRs of the heavy chain variable domain, SEQ ID NOs: 1-3, and CDRs of the light chain variable domain, SEQ ID NOs: 4-6 as defined by Kabat numbering. The CDRs can also be defined by Chothia system. Under the Chothia numbering, the CDR sequences of the heavy chain variable domain are set forth in SEQ ID NOs: 19-21 and CDR sequences of the light chain variable domain are set forth in SEQ ID NOs: 22-24.

Similarly, the CDR sequences for 6A4, and 6A4 derived antibodies include CDRs of the heavy chain variable domain, SEQ ID NOs: 7-9, and CDRs of the light chain variable domain, SEQ ID NOs: 10-12, as defined by Kabat numbering. Under Chothia numbering, the CDR sequences of the heavy chain variable domain are set forth in SEQ ID NOs: 25-27, and CDRs of the light chain variable domain are set forth in SEQ ID NOs: 28-30.

The CDR sequences for 12B5, and 12B5 derived antibodies include CDRs of the heavy chain variable domain, SEQ ID NOs: 13-15, and CDRs of the light chain variable domain, SEQ ID NOs: 16-18, as defined by Kabat numbering. Under Chothia numbering, the CDR sequences of the heavy chain variable domain are set forth in SEQ ID NOs: 31-33, and CDRs of the light chain variable domain are set forth in SEQ ID NOs: 34-36.

The amino acid sequence for the heavy chain variable region of 2F8 antibody is set forth in SEQ ID NO: 65. The amino acid sequence for the light chain variable region of 2F8 antibody is set forth in SEQ ID NO: 37.

The amino acid sequence for the heavy chain variable region of 6A4 antibody is set forth in SEQ ID NO: 38. The amino acid sequence for the light chain variable region of 6A4 antibody is set forth in SEQ ID NO: 39.

The amino acid sequence for the heavy chain variable region of 12B5 antibody is set forth in SEQ ID NO: 40. The amino acid sequence for the light chain variable region of 12B5 antibody is set forth in SEQ ID NO: 41.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 65, 38, or 40. In some embodiments, the light chain variable region is at least 80%, 85%, 90%, 91%, 92%, 93%, 94%, 95%, 96%, 97%, 98%, or 99% identical to SEQ ID NO: 37, 39, or 41. The heavy chain variable region sequence can be paired with the corresponding light chain variable region sequence, and together they bind to CD40.

Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 1-3, SEQ ID NOs: 7-9, SEQ ID NOs: 13-15, SEQ ID NOs: 19-21, SEQ ID NOs: 25-27, and SEQ ID NOs: 31-33; and/or one, two, or three light chain variable region CDRs selected from the group of SEQ ID NOs: 4-6, SEQ ID NOs: 10-12, SEQ ID NOS: 16-18, SEQ ID NOs: 22-24, SEQ ID NOs: 28-30, and SEQ ID NOs: 34-36.

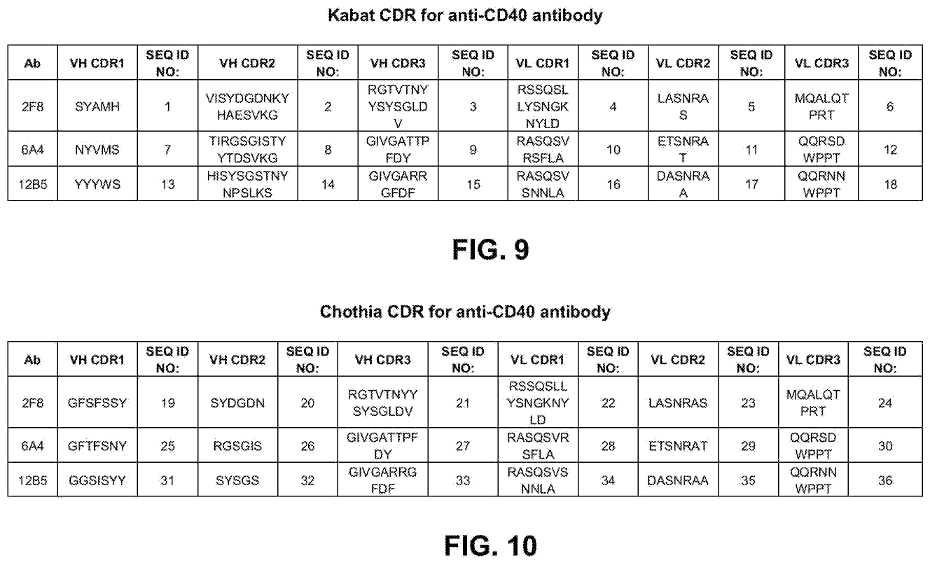

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 9 (Kabat CDR) and FIG. 10 (Chothia CDR).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a light chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a light chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a light chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a light chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a light chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination of Kabat and Chothia numbering scheme.

The disclosure also provides antibodies or antigen-binding fragments thereof that bind to CD40. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 65, and the selected VL sequence is SEQ ID NO: 37. In some embodiments, the selected VH sequence is SEQ ID NO: 38 and the selected VL sequence is SEQ ID NO: 39. In some embodiments, the selected VH sequence is SEQ ID NO: 40, and the selected VL sequence is SEQ ID NO: 41.

The disclosure also provides antibodies or antigen-binding fragments thereof that can compete with the antibodies described herein. In some aspects, the antibodies or antigen-binding fragments can bind to the same epitope as the antibodies described herein.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

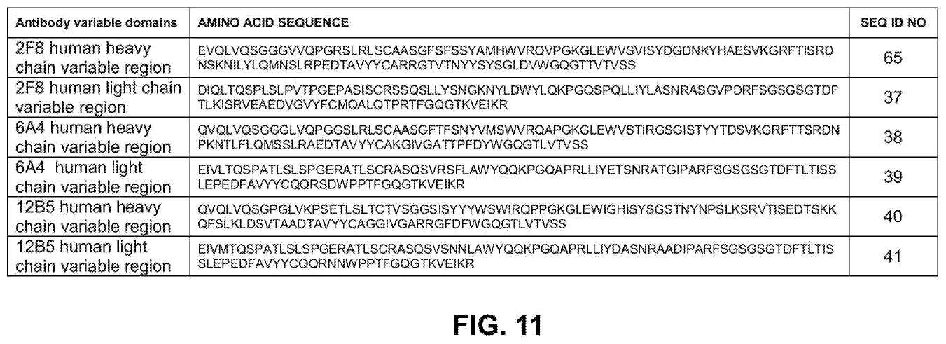

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 9 or FIG. 10, or have sequences as shown in FIG. 11. When the polypeptides are paired with corresponding polypeptide (e.g., a corresponding heavy chain variable region or a corresponding light chain variable region), the paired polypeptides bind to CD40 (e.g., human CD40).

The anti-CD40 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CD40 will retain an ability to bind to CD40. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antibody or antigen-binding fragment thereof described herein recognizes an endogenous CD40 or a recombinant CD40. In some embodiments, the antibody or antigen-binding fragment thereof described herein recognizes human CD40.

In some embodiments, the half-life of the antibody or antigen-binding fragment thereof described herein in wild-type mice (e.g., C57BL/6 mice) is at least 10 days, at least 11 days, at least 12 days, at least 13 days, at least 14 days, at least 15 days, at least 16 days, at least 17 days, or at least 18 days. In some embodiments, the half-life of the antibody or antigen-binding fragment thereof described herein in CD40 gene humanized mice (e.g., hCD40 mice) is at least 1 day, at least 2 days, at least 3 days, at least 4 days, or at least 5 days.

In some embodiments, the clearance rate (CL) of the antibody or antigen-binding fragment thereof described herein in wild-type mice (e.g., C57BL/6 mice) is less than 7 ml/day/kg, less than 6 ml/day/kg, less than 5 ml/day/kg, or less than 4 ml/day/kg. In some embodiments, the clearance rate (CL) of the antibody or antigen-binding fragment thereof described herein in CD40 gene humanized mice (e.g., hCD40 mice) is less than 15 ml/day/kg, less than 14 ml/day/kg, less than 13 ml/day/kg, or less than 12 ml/day/kg.

In some embodiments, the half-life of the antibody or antigen-binding fragment thereof described herein in FcRn gene humanized mice (e.g., hFcRn mice) is at least 6 days, at least 7 days, at least 8 days, at least 9 days, at least 10 days, at least 11 days, at least 12 days, at least 13 days, or at least 14 days. In some embodiments, the clearance rate (CL) of the antibody or antigen-binding fragment thereof described herein in FcRn gene humanized mice (e.g., hFcRn mice) is less than 16 ml/day/kg, less than 15 ml/day/kg, less than 14 ml/day/kg, less than 13 ml/day/kg, less than 12 ml/day/kg, less than 11 ml/day/kg, less than 10 ml/day/kg, less than 9 ml/day/kg, less than 8 ml/day/kg, or less than 7 ml/day/kg.

In some embodiments, the antibody or antigen-binding fragment thereof described herein can enter a cell expressing an endogenous, recombinant, or human CD40. In some embodiments, the antibody or antigen-binding fragment thereof described herein can enter a cell through endocytosis. In some embodiments, the antibody or antigen-binding fragments described herein enters at least 10%, at least 20%, at least 30%, at least 40%, at least 50%, at least 60%, at least 70%, at least 80%, at least 90%, or 100% of the cells (e.g., CD40-expressing cells) through endocytosis.

In some embodiments, the antibody or antigen-binding fragment thereof described herein exhibits immune-stimulating effects. In some embodiments, the antibody or antigen-binding fragment thereof described herein exhibits immune-suppressing effects. In some embodiments, the antibody or antigen-binding fragment thereof described herein suppresses one or more immune functions (e.g., antigen-induced antibody production) to less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, or less than 1% as compared to the same immune function when the antibody or antigen-binding fragment thereof is not administered.

In some embodiments, the immune-suppressing effects of the antibody or antigen-binding fragment thereof described herein is reversible. In some embodiments, the immune-suppressing effects of the antibody or antigen-binding fragment thereof described herein is irreversible. In some embodiments, immune functions (e.g., T-cell dependent humoral immune function) of a subject (e.g., a mouse) are recovered after at least 10 days, at least 15 days, at least 20 days, at least 25 days, at least 30 days, at least 32 days, at least 35 days, at least 40 days, at least 45 days, or at least 60 days after the subject is administered with the antibody or antigen-binding fragment thereof. In some embodiments, immune functions (e.g., T-cell dependent humoral immune function) of a subject (e.g., a mouse) are recovered to at least 50%, at least 60%, at least 70%, at least 80%, at least 90%, at least 95%, or more as compared to the same immune function before the subject is administered with the antibody or antigen-binding fragment thereof.

In some embodiments, the immune-suppressing effects of the antibody or antigen-binding fragment thereof does not reduce the percentage of CD20⁺/CD19⁺ cells in an organ (e.g., spleen) of the immune system of a subject (e.g., a mouse).

In some embodiments, the antibody or antigen-binding fragment thereof saturates CD40 receptors at a concentration at about or less than 0.1 μg/ml, about 0.2 μg/mL, about 0.3 μg/mL, 0.4 μg/mL, 0.5 μg/mL, 1 μg/mL, 2 μg/mL, 5 μg/mL, 10 μg/mL.

In some embodiments, the antibody or antigen-binding fragment thereof described herein decreases CD154 binding to CD40 to less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, or less than 1%.

In some embodiments, the percentage of CD40 receptor occupancy (RO %) of the antibody or antigen-binding fragment thereof is at least 10%, at least 20%, at least 30%, at least 40%, at least 50%, at least 60%, at least 70%, at least 80%, at least 90%, or 100%. In some embodiments, the antibody or antigen-binding fragment thereof decreases percentage of activated B cells to less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, or less than 1% as compared to the percentage of activated B cells when the antibody or antigen-binding fragment thereof is not administered.

### Antibody Characteristics

The antibodies or antigen-binding fragments thereof described herein can block the binding between CD40 and CD40 ligands (e.g., CD154).

The antibodies or antigen-binding fragments thereof as described herein can be CD40 agonist or antagonist. In some embodiments, by binding to CD40, the antibody can inhibit CD40 signaling pathway. In some embodiments, the antibody can upregulate immune response or downregulate immune response.

In some embodiments, the antibodies or antigen-binding fragments thereof as described herein can increase immune response, activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds. In some embodiments, the antibodies or antigen-binding fragments thereof as described herein can decrease the activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds.

In some implementations, the antibody (or antigen-binding fragments thereof) specifically binds to CD40 (e.g., human CD40, monkey CD40 (e.g., rhesus macaques, Macaca fascicularis), dog CD40, mouse CD40, and/or chimeric CD40) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, and surface plasmon resonance (SPR). In some embodiments, the antibody binds to human CD40 (SEQ ID NO: 42), mouse CD40 (SEQ ID NO: 50), and/or chimeric CD40 (SEQ ID NO: 49). In some embodiments, the antibody does not bind to human CD40 (SEQ ID NO: 42), mouse CD40 (SEQ ID NO: 50), and/or chimeric CD40 (SEQ ID NO: 49). In some embodiments, the antibody binds to FcRn (SEQ ID NO: 51).

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$T G I ( \% ) = [ 1 - ( Ti - T 0 ) / ( Vi - V 0 ) ] \times 100 \%$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen-binding fragments thereof as described herein are CD40 antagonist. In some embodiments, the antibodies or antigen binding fragments decrease CD40 signal transduction in a target cell that expresses CD40.

In some embodiments, the antibodies or antigen binding fragments can enhance APC (e.g., DC cell) function, for example, inducing surface expression of costimulatory and MHC molecules, inducing production of proinflammatory cytokines, and/or enhancing T cell triggering function.

In some embodiments, the antibodies or antigen binding fragments can bind to tumor cells that express CD40. In some embodiments, the antibodies or antigen binding fragments can induce complement mediated cytotoxicity (CMC) and/or antibody dependent cellular cytoxicity (ADCC), and kill the tumor cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments can induce antibody dependent cellular cytoxicity (ADCC). In some embodiments, the antibodies or antigen binding fragments cannot induce antibody dependent cellular cytoxicity (ADCC). In some embodiments, the antibodies or antigen binding fragments can induce complement mediated cytotoxicity (CMC). In some embodiments, the antibodies or antigen binding fragments cannot induce complement mediated cytotoxicity (CMC).

In some embodiments, the Fc region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments do not have a functional Fc region. For example, the antibodies or antigen binding fragments are Fab, Fab′, F(ab′)₂, and Fv fragments. In some embodiments, the Fc region has LALA mutations (L234A and L235A mutations according to EU numbering), or LALA-PG mutations (L234A, L235A, P329G mutations according to EU numbering). In some embodiments, the Fc region has FLAA mutations (F234A and L235A according to EU numbering). In some embodiments, the Fc has SI mutations (S239D and 1332E mutations according to EU numbering). In some embodiments, the Fc has N297A mutation according to EU numbering. In some embodiments, the Fc has YTE mutations (M252Y, S254T and T256E according to EU numbering).

In some embodiments, the antibodies or antigen binding fragments described herein include an amino acid sequence that is at least 80%, at least 85%, at least 90%, at least 95% or 100% identical to any one of SEQ ID NOs: 52-59.

In some embodiments, the antibodies or antigen binding fragments described herein can inhibit PMBC proliferation, e.g., to less than 90%, less than 80%, less than 70%, less than 60%, less than 50%, less than 40%, or less than 30% as compared to an isotype control antibody (e.g., hIgG1).

In some embodiments, the antibodies or antigen binding fragments described herein can inhibit B cell proliferation and/or activation of B cells. For example, the percentage of activated B cell subsets in PMBC cells, upon treatment of the antibodies or antigen binding fragments described herein, can be reduced to less than 90%, less than 80%, less than 70%, less than 60%, less than 50%, less than 40%, less than 30%, less than 20%, less than 10%, or less than 5% as compared to that upon treatment of an isotype control antibody (e.g., hIgG1).

### Methods of Making Anti-CD40 Antibodies

An isolated fragment of human CD40 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of CD40 and encompasses an epitope of the protein such that an antibody raised against the peptide forms a specific immune complex with the protein. As described above, the full length sequence of human CD40 (SEQ ID NO: 42) is known in the art. In some embodiments, an Fc-tagged or His-tagged human CD40 protein is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human CD40). The preparation can further include an adjuvant, such as Freund's complete or incomplete adjuvant, or a similar immunostimulatory agent.

Polyclonal antibodies can be prepared as described above by immunizing a suitable subject with a CD40 polypeptide, or an antigenic peptide thereof (e.g., part of CD40) as an immunogen. The antibody titer in the immunized subject can be monitored over time by standard techniques, such as with an enzyme-linked immunosorbent assay (ELISA) using the immobilized CD40 polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., CD40. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-CD40 antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chains. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant regions (e.g., IgG1, IgG2, IgG3, and IgG4). In some embodiments, the constant region has a sequence that is at least at least 80%, 85%, 90%, 91%, 92%, 93%, 94%, 95%, 96%, 97%, 98%, or 99% identical to SEQ ID NO: 52, 53, 54, 55, 56, 57, 58, or 59.

Identity or homology with respect to an original sequence is usually the percentage of amino acid residues present within the candidate sequence that are identical with a sequence present within the human, humanized, or chimeric anti-CD40 antibody or fragment, after aligning the sequences and introducing gaps, if necessary, to achieve the maximum percent sequence identity, and not considering any conservative substitutions as part of the sequence identity.

Additional modifications to the anti-CD40 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-CD40 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing disorders associated with an abnormal or unwanted immune response, e.g., an autoimmune disorder, e.g., by affecting the functional properties of the APC cells (e.g., by blocking the interaction between CD40 and CD40L). These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-CD40 antibodies or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has dermatological disorders, liver disease (e.g., cirrhosis), Hidradenitis, experimental autoimmune encephalomyelitis. In some embodiments, the subject has renal disease, lupus, Sjogren's syndrome, ulcerative colitis, psoriasis, Allergic Dermatitis, Atopic Dermatitis, Hidradenitis suppurativa, Immune Thrombocytopenia (ITP), or other inflammatory arthritis. In some embodiments, the subject has multiple sclerosis or myasthenia gravis. In some embodiments, the subject has Crohn's disease, ulcerative colitis or type 1 diabetes. In some embodiments, the subject has autoimmune thyroid disease, Grave's disease, multiple sclerosis, psoriasis, inflammatory bowel disease (e.g., Crohn's Disease (CD) and ulcerative colitis), rheumatoid arthritis, Sjögren's syndrome, autoimmune nephritis, or systemic lupus erythematosus. A relationship of CD40 and various autoimmune diseases are described e.g., in Peters et al., “CD40 and autoimmunity: the dark side of a great activator.” Seminars in immunology. Vol. 21. No. 5. Academic Press, 2009, and Karnell, Jodi L., et al. “Targeting the CD40-CD40L pathway in autoimmune diseases: Humoral immunity and beyond.” Advanced drug delivery reviews (2018); Albach, et al. “Safety, pharmacokinetics and pharmacodynamics of single rising doses of BI 655064, an antagonistic anti-CD40 antibody in healthy subjects: a potential novel treatment for autoimmune diseases.” European journal of clinical pharmacology 74.2 (2018): 161-169; each of which are incorporated herein by reference in the entirety.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, an anti-ICOS antibody, an anti-CD27 antibody, an anti-OX40 antibody, an anti-4-1BB antibody, and/or an anti-GITR antibody. In some embodiments, the anti-CD40 antibody or antigen-binding fragment thereof described herein can be administered together with an anti-PD-1 antibody and an anti-TIGIT antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Human Anti-CD40 Antibodies

To generate antibodies against human CD40, RenMab mice or RenLite™ mice were immunized with human CD40. Anti-CD40 antibodies were made by the methods as described below.

RenMab™ mice have both a humanized heavy chain immunoglobulin locus and a humanized kappa chain immunoglobulin locus. The heavy chain immunoglobulin locus is a region on the chromosome that contains genes for the heavy chains of antibodies. The locus includes IGHV (variable), IGHD (diversity), IGHJ (joining), and heavy chain constant domain genes. The kappa chain immunoglobulin locus is a region on the chromosome that contains genes that encode the light chains of antibodies (kappa chain). The kappa chain immunoglobulin locus includes IGKV (variable), IGKJ (joining), and light chain constant domain genes. Detailed descriptions regarding RenMab™ mice can be found in PCT/CN2020/075698, which is incorporated herein by reference in its entirety.

RenLite™ mice can be used as a genetically-engineered model with complete humanization in the variable region of the heavy chains, while maintaining a fully-humanized common light chain strategically engineered into the antibody gene. Details of RenLite™ mice can be found, e.g., in PCT/CN2021/097652, which is incorporated herein by reference in its entirety.

### Immunization of Mice

RenMab™ mice or RenLite™ mice were immunized with Fc-tagged human CD40 protein (hCD40-Fc, Beijing ACROBiosystems Co. Ltd., Cat #: CD0-H5253) or dog CD40 (dCD40-Fc, Sino Biological Inc., Cat #: 70105-D02H). Specifically, hCD40-Fc or dCD40-Fc was emulsified with adjuvant and injected at heel, neck or tail base of the mice. The mice were immunized for a total of 3 times: complete Freund's adjuvant (CFA) was used for the first immunization, and incomplete Freund's adjuvant (IFA) was used for the second and third immunizations. The interval between the first and second immunizations was two weeks, and the interval between the second and third immunizations was two weeks. Orbital blood was collected 1 week after the third immunization, which was analyzed for antibody titer using ELISA. Two week later, mice with high titers were selected and injected with hCD40-His (Human CD40 protein having a His-Tag, Beijing ACROBiosystems Co. Ltd., Cat #: CD0-H5228) or dCD40-His (CD40 Protein, Canine, Recombinant (His Tag), Sino Biological Inc., Cat #: 70105-D08H) via tail vein.

In another experiment, RenMab™ mice or RenLite™ mice were immunized by injecting an expression plasmid encoding human CD40 and/or dog CD40 into the tibialis anterior muscle of the mice (by intramuscular (i.m.) injection). The mice were injected for at least four times with at least 14 days between each injection. Blood was collected seven days after the last immunization and the serum was tested for antibody titer by ELISA.

Procedures to enhance immunization were also performed at least seven days after immunization (either by injecting the expression plasmid or by injecting the proteins). The mice were also injected (by intraperitoneal administration) with CHO-S-hCD40 (transfected CHO-S cells expressing human CD40 protein) or CHO-S-dCD40 (transfected CHO-S cells expressing dog CD40 protein) for pulse immunization. Immune system organs (e.g., bone marrow, lymph nodes, and spleen) were then collected five days after the injection.

Antigen-specific B cells were also isolated directly from the immunized mice without fusion with myeloma cells. The antibody light and heavy chain variable region sequences were directly obtained from the antigen-specific B cells. For example, single cell technology (e.g., Beacon® Optofluidic System, Berkeley Lights Inc.) was used to screen plasma cells that secrete antigen-specific monoclonal antibodies. The antibody variable region sequences were obtained using reverse transcription and PCR-based sequencing. Antibodies were expressed by transfecting cells with vectors including the antibody variable region sequences. FACS was used to verify the binding between the antibody and CD40. Exemplary anti-CD40 antibodies obtained by this method include: 2F8, 6A4, 15B4, 16D5 and 12B5.

The antibodies were named following the rules below. For example, when the heavy chain variable region (VH) and light chain variable region (VL) of the 2F8 antibody were connected to constant regions of human IgG1, the antibody was named 2F8-IgG1. Similarly, 2F8-IgG2 and 2F8-IgG4 were generated, which included the same VH and VL sequences of 2F8, but the constant regions were from human IgG2 and IgG4 subtypes, respectively.

The constant regions can also include mutations. For example, LALA mutations (L234A and L235A according to EU numbering), N297A, YTE mutations (M252Y, S254T and T256E according to EU numbering), FLAA mutations (F234A and L235A according to EU numbering), or SI mutations (S239D and 1332E according to EU numbering) were introduced into the Fc region of 2F8-IgG1, generating 2F8-IgG1-LALA, 2F8-IgG1-N297A, 2F8-IgG1-YTE, 2F8-IgG1-FLAA, and 2F8-IgG1-SI, respectively. YTE mutations were also introduced into the Fc region of 2F8-IgG1-LALA or 2F8-IgG1-N297A, generating 2F8-IgG1-LALA-YTE or 2F8-IgG1-N297A-YTE respectively. Antibodies with different constant region sequences were also produced for 6A4, 15B4, 16D5 and 12B5.

The amino acid sequences of heavy chain CDRs 1, 2, 3, and light chain CDRs 1, 2, 3 for 2F8 are shown in SEQ ID NOs: 1-6 (Kabat numbering) or SEQ ID NOs: 19-24 (Chothia numbering), respectively. The human heavy chain variable region and human light chain variable region for 2F8 are shown in SEQ ID NO: 65 and SEQ ID NO: 37, respectively.

The amino acid sequences of heavy chain CDRs 1, 2, 3, and light chain CDRs 1, 2, 3 for 6A4 are shown in SEQ ID NOs: 7-12 (Kabat numbering) or SEQ ID NOs: 25-30 (Chothia numbering), respectively. The human heavy chain variable region and human light chain variable region for 6A4 are shown in SEQ ID NO: 38 and SEQ ID NO: 39, respectively.

The amino acid sequences of heavy chain CDRs 1, 2, 3, and light chain CDRs 1, 2, 3 for 12B5 are shown in SEQ ID NOs: 13-18 (Kabat numbering) or SEQ ID NOs: 31-36 (Chothia numbering), respectively. The human heavy chain variable region and human light chain variable region for 12B5 are shown in SEQ ID NO: 40 and SEQ ID NO: 41, respectively.

### Antibody Preparation

The positive antibody sequences in the sequence verification stage were subjected to plasmid extraction and transfection into a 100 ml system. The expression supernatants were collected after 10-13 days of cell culture and then subject to affinity chromatography. The antibody samples obtained were used in the following in vitro testing and screening.

### Example 2. Binding Affinity of Anti-CD40 Antibodies

Bleselumab (VH SEQ ID NO: 43, VL SEQ ID NO: 44; HC SEQ ID NO: 66, LC SEQ ID NO: 60) is a fully human IgG4 monoclonal antibody targeting CD40, which was approved for treatment of lupus.

The binding affinity of the anti-CD40 antibodies 2F8-IgG4-FLAA, 6A4-IgG4-FLAA, 12B5-IgG4-FLAA and Bleselumab analog to human CD40 (hCD40), monkey (Macaca fascicularis) CD40 (fasCD40), or dog CD40 (dCD40) was measured using surface plasmon resonance (SPR) using Biacore™ (Biacore, Inc., Piscataway N.J.) 8K biosensor equipped with pre-immobilized Protein A sensor chips.

Specifically, hCD40-His, fasCD40-His (Cynomolgus CD40/TNFRSF5 Protein, His Tag, Beijing ACRO Biosystems Co. Ltd., Cat #: CD0-C52H6), or dCD40-His was diluted to 200 nM, 100 nM, 50 nM, 25 nM, 6.25 nM, 3.125 nM or 0 nM with 1×HBS-EP+ buffer (PH 7.4) and then injected into the Biacore™ 8K biosensor at 10 μl/min for about 50 seconds to meet the required capture level (e.g., about 100 response units (RU)). Purified anti-CD40 antibodies at concentrations of 1 μg/ml with 1×HBS-EP+ buffer (PH 7.4) were then injected at 1 μl/min for 50 seconds. Dissociation was monitored for 400 seconds. The chip was regenerated after the last injection of each titration with a glycine solution (pH 2.0) at 30 μl/min for 30 seconds.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

The results for the tested antibodies are summarized in the table below. The results showed that anti-CD40 antibodies 2F8-IgG4-FLAA and 12B5-IgG4-FLAA specifically binds to human, monkey, and dog CD40. 6A4-IgG4-FLAA specifically binds to human and monkey CD40.

TABLE 1

| Anti-CD40 | Analyte 1 | Kₒₙ | K_{off} | KD |
|---|---|---|---|---|
| antibodies | Solution | (1/Ms) | (1/s) | (M) |
| 2F8-IgG4-FLAA | hCD40-His | 6.54E+05 | 2.29E−04 | 3.51E−10 |
| 2F8-IgG4-FLAA | fasCD40-His | 9.89E+05 | 2.97E−04 | 3.01E−10 |
| 2F8-IgG4-FLAA | dCD40-His | 5.13E+05 | 2.66E−03 | 5.18E−09 |
| 6A4-IgG4-FLAA | hCD40-His | 3.09E+05 | 5.11E−04 | 1.66E−09 |
| 6A4-IgG4-FLAA | fasCD40-His | 2.88E+05 | 1.49E−02 | 5.17E−08 |
| 6A4-IgG4-FLAA | dCD40-His | — | — | — |
| 12B5-IgG4-FLAA | hCD40-His | 5.29E+05 | 3.89E−04 | 7.35E−10 |
| 12B5-IgG4-FLAA | fasCD40-His | 5.35E+05 | 2.65E−04 | 4.95E−10 |
| 12B5-IgG4-FLAA | dCD40-His | 4.01E+05 | 1.29E−02 | 3.21E−08 |
| Bleselumab analog | hCD40-His | 4.29E+05 | 6.82E−04 | 1.59E−09 |
| Bleselumab analog | fasCD40-His | 2.70E+05 | 9.07E−04 | 3.35E−09 |
| Bleselumab analog | dCD40-His | — | — | — |

### Example 3. Jurkat-Luc-hCD40 Reporter Cell Activation Assays

Iscalimab (VH SEQ ID NO: 45, VL SEQ ID NO: 46; HC SEQ ID NO: 61, LC SEQ ID NO: 62) is a fully human IgG1 antibody that blocks CD40 signaling pathway, which was developed by Novartis and XOMA, for the treatment of Sjogren's syndrome.

BI655064 (VH SEQ ID NO: 47, VL SEQ ID NO: 48; HC SEQ ID NO: 63, LC SEQ ID NO: 64) is a humanized, purely antagonistic anti-CD40 IgG1 monoclonal antibody, which was developed by Boehringer Ingelheim, for the treatment of rheumatoid arthritis (RA) and lupus nephritis, etc.

The experiment was performed to test whether anti-CD40 antibodies 2F8-IgG4-FLAA, 6A4-IgG4-FLAA, 15B4-IgG4-FLAA, 12B5-IgG4-FLAA, BI655064 analog, Isalimab analog and Bleselumab analog can block the CD40 signaling pathway.

CHO-K1-FcγRIIB cells (Promega, Cat #: CS1979A09) were seeded in a 96-well plate (cell density 4×10⁴ cells/well) and incubated at 37° C. overnight. The tested antibodies were serially diluted (3-fold) with the highest concentration at 6 μg/ml. Human CD40 ligand CD40L-His (Human CD40 Ligand/TNFSF5 Protein, His, Flag Tag (active trimer) (MALS verified), Beijing ACROBiosystems Co. Ltd., Cat #: CDL-H52Db) was serially diluted with the highest concentration at 3 μg/ml. Jurkat-Luc-hCD40 cells (transfected Jurkat-Luc cells expressing human CD40 protein) were seeded in the 96-well plate (cell density 5×10⁵ cells/well), and then 25 μl of the serially diluted antibody solution and/or human CD40 ligand were added to each well. The working concentrations of the antibodies were 0.5 μg/ml, 1 μg/ml, and 2 μg/ml. The above 96-well plate was incubated in a 37° C. incubator for 6 hours. After the incubation, the plate was taken out, and 75 μl of Bio-lite™ Luciferase Assay Reagent (Vazyme Biotech Co., Ltd., Cat #: DD1201-02-AB) was added to each well. The plate was incubated at room temperature for 5-10 minutes, and then placed in a luminescence detector to detect the fluorescence signal.

When the concentration of all the anti-CD40 antibodies decreased, the fluorescence signal (indicating cells binding to CD40 ligands) increased, suggesting that the binding between human CD40 and human CD40 Ligand was blocked by the anti-CD40 antibodies.

### Example 4: Immunosuppressive Effects of Anti-CD40 Antibodies In Vivo Immunosuppressive Effects of Anti-CD40 Antibodies in hCD40 Mice

A CD40 gene humanized mouse model was generated to express a chimeric CD40 protein (SEQ ID NO: 49) wherein a part of the extracellular region of the mouse CD40 protein was replaced with the corresponding human CD40 extracellular region. Specifically, amino acids 20-192 of mouse CD40 (SEQ ID NO: 50) were replaced with amino acids 20-192 of human CD40 (SEQ ID NO: 42). The humanized mouse model (hCD40 mice) provides a tool for testing new therapeutic treatments in a clinical setting by significantly decreasing the difference between clinical outcome in human and in laboratory mice expressing mouse CD40. A detailed description of CD40 gene humanized mouse model can be found in PCT/CN2018/091845, which is incorporated herein by reference in its entirety.

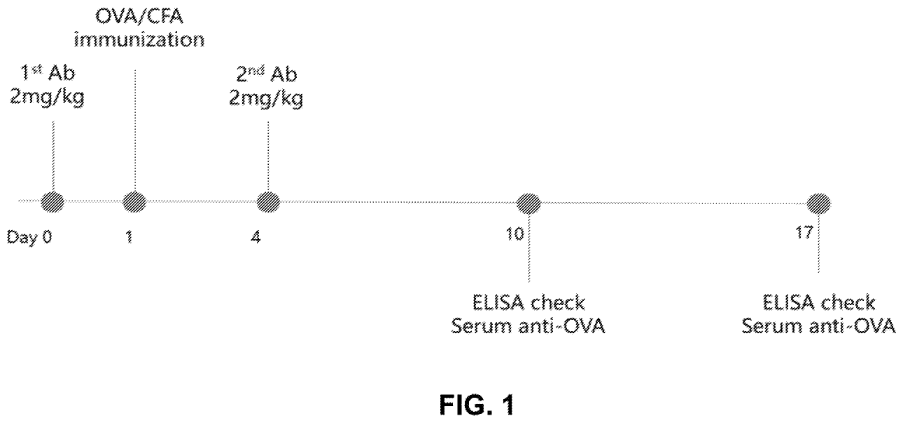

Experiments were performed to analyze the effects of the human anti-CD40 antibodies on immune responses. Ovalbumin (OVA) was used as an antigen to stimulate immune responses in the hCD40 mice. Briefly, hCD40 mice (6-8 weeks old) were placed into one control group and six treatment groups (5 mice per group). The treatment group mice were randomly selected for intraperitoneal (i.p.) administration of anti-CD40 antibodies Bleselumab analog (G2), 12B5-IgG4-FLAA (G3), 16D5-IgG4-FLAA (G4), 2F8-IgG4-FLAA (G5), 6A4-IgG4-FLAA (G6) and 15B4-IgG4-FLAA (G7), respectively, each at a dose level of 2 mg/kg. The control group mice (G1) were injected with an equal volume of phosphate buffered saline (PBS). A total of two administrations were performed on Day 0 (grouping day) and Day 4 (4 days after grouping), respectively. Ovalbumin (OVA) was diluted to 1 mg/ml by PBS, mixed with CFA at a volume ratio of 1:1, and the immunization volume was 200 μl/mouse. Mice were administered with the OVA/CFA mixture intraperitoneally on Day 1 (1 day after grouping). Serum was collected on Day 10 and Day 17 from each animal and subjected to ELISA analysis. FIG. 1 shows the experimental scheme. OVA was pre-coated on the ELISA plate. Goat Anti-Mouse IgG H&L (HRP) (Abcam, Cat #: ab97265) was used for ELISA analysis. Details of the administration scheme are shown in the table below.

TABLE 2

| | No. of | | Dosage | | | Total No. of |
|---|---|---|---|---|---|---|
| Group | mice | Antibodies | (mg/kg) | Route | Frequency | administration |
| G1 | 5 | PBS | — | i.p. | BIW | 2 |
| G2 | 5 | Bleselumab analog | 2 | i.p. | BIW | 2 |
| G3 | 5 | 12B5-IgG4-FLAA | 2 | i.p. | BIW | 2 |
| G4 | 5 | 16D5-IgG4-FLAA | 2 | i.p. | BIW | 2 |
| G5 | 5 | 2F8-IgG4-FLAA | 2 | i.p. | BIW | 2 |
| G6 | 5 | 6A4-IgG4-FLAA | 2 | i.p. | BIW | 2 |
| G7 | 5 | 15B4-IgG4-FLAA | 2 | i.p. | BIW | 2 |

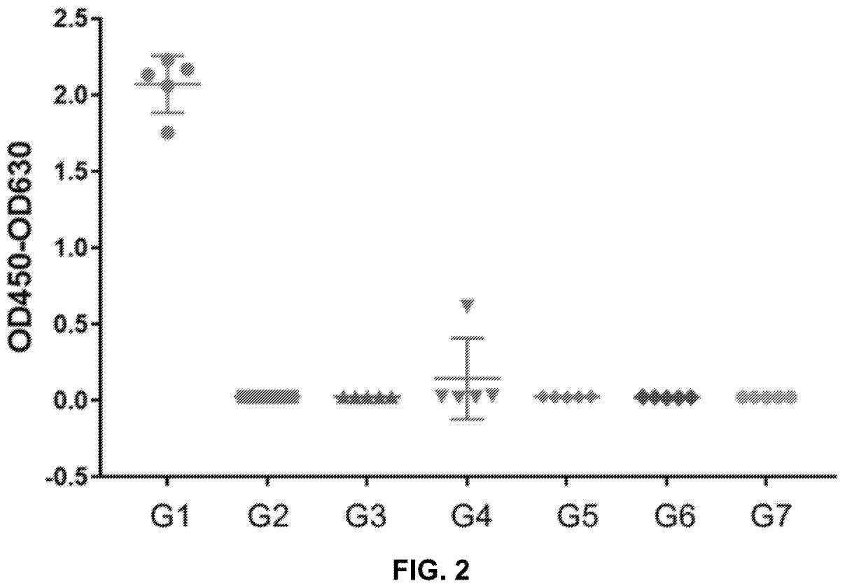

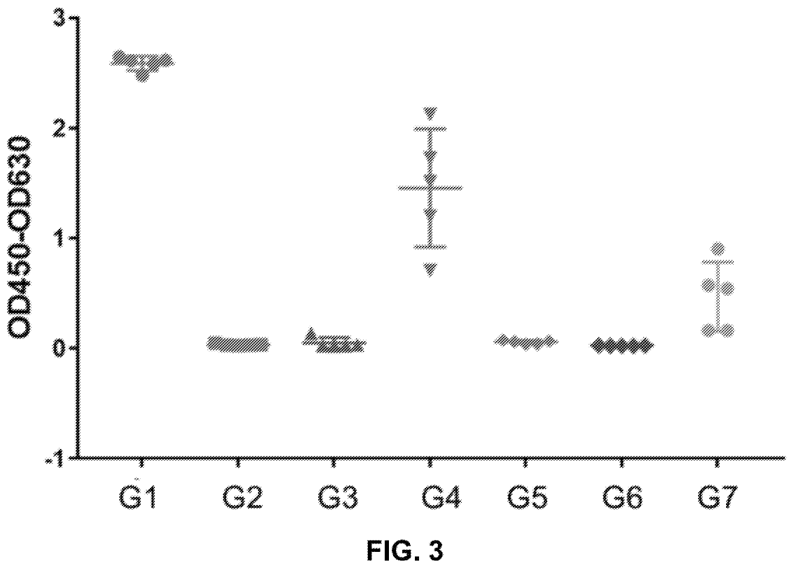

As shown in FIG. 2, the ELISA results for the serum collected on Day 10 showed that anti-CD40 antibodies Bleselumab analog (G2), 12B5-IgG4-FLAA (G3), 16D5-IgG4-FLAA (G4), 2F8-IgG4-FLAA (G5), 6A4-IgG4-FLAA (G6) and 15B4-IgG4-FLAA (G7) reduced immune response in hCD40 mice as compared to PBS (G1). FIG. 3 shows the ELISA results for the serum collected on Day 17. The results on Day 17 were similar to those obtained on Day 10. Specifically, Bleselumab analog (G2), 12B5-IgG4-FLAA (G3), 2F8-IgG4-FLAA (G5) and 6A4-IgG4-FLAA (G6) reduced immune response with a lower OD than 16D5-IgG4-FLAA (G4) and 15B4-IgG4-FLAA (G7). It is contemplated that more antibodies exhibited reduced immune response on Day 10 because of increased antibody degradation on Day 17.

Immunosuppressive Effects of Anti-CD40 Antibodies in hCD40/hFcRn Mice

A CD40/FcRn double-gene humanized mouse model was also generated by crossing the hCD40 mice with FcRn gene humanized mice (hFcRn mice). The FcRn gene humanized mice were engineered to express a human FcRn protein (SEQ ID NO: 51). A detailed description of the FcRn gene humanized mice and CD40/FcRn double-gene humanized mice (hCD40/hFcRn mice) can be found in PCT/CN2022/075057, which is incorporated herein by reference in its entirety.

Similar to the above experiments, the pharmacokinetics (PK) and in vivo efficacy of the anti-CD40 antibodies Bleselumab analog, 12B5-IgG1-LALA-YTE, and 2F8-IgG1-LALA-YTE were tested in hCD40/hFcRn mice. In treatment groups (G2-G10), the hCD40/hFcRn mice were injected with 1-4 mg/kg (i.e., 1 mg/kg, 2 mg/kg, or 4 mg/kg) of the anti-CD40 antibodies Bleselumab analog, 12B5-IgG1-LALA-YTE, and 2F8-IgG1-LALA-YTE, respectively, by intraperitoneal (i.p.) administration on Day 0 (grouping day). The control group mice (G1) were injected with an equal volume of PBS. Mice were administered with the OVA/CFA mixture intraperitoneally on Day 1 (1 day after grouping). Serum was collected 4 hours after antibodies administration, and on Day 1, Day 5, Day 10, Day 17 and Day 21 from each animal. The collected serum was then subjected to PK analysis. Serum collected on Day 10, Day 17 and Day 21 from each animal was also subjected to ELISA analysis. Details of the administration scheme are shown in the table below.

TABLE 3

| | No. of | | Dosage | | | Total No. of |
|---|---|---|---|---|---|---|
| Group | mice | Antibodies | (mg/kg) | Route | Frequency | administration |
| G1 | 5 | PBS | — | i.p. | Day 0 | 1 |
| G2 | 5 | Bleselumab analog | 4 | i.p. | Day 0 | 1 |
| G3 | 5 | Bleselumab analog | 2 | i.p. | Day 0 | 1 |
| G4 | 5 | Bleselumab analog | 1 | i.p. | Day 0 | 1 |
| G5 | 5 | 12B5-IgG1-LALA-YTE | 4 | i.p. | Day 0 | 1 |
| G6 | 5 | 12B5-IgG1-LALA-YTE | 2 | i.p. | Day 0 | 1 |
| G7 | 5 | 12B5-IgG1-LALA-YTE | 1 | i.p. | Day 0 | 1 |
| G8 | 5 | 2F8-IgG1-LALA-YTE | 4 | i.p. | Day 0 | 1 |
| G9 | 5 | 2F8-IgG1-LALA-YTE | 2 | i.p. | Day 0 | 1 |
| G10 | 5 | 2F8-IgG1-LALA-YTE | 1 | i.p. | Day 0 | 1 |

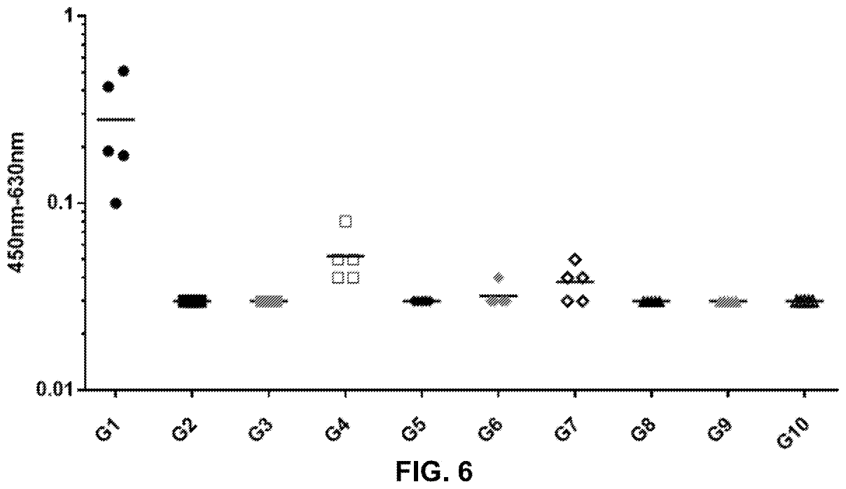

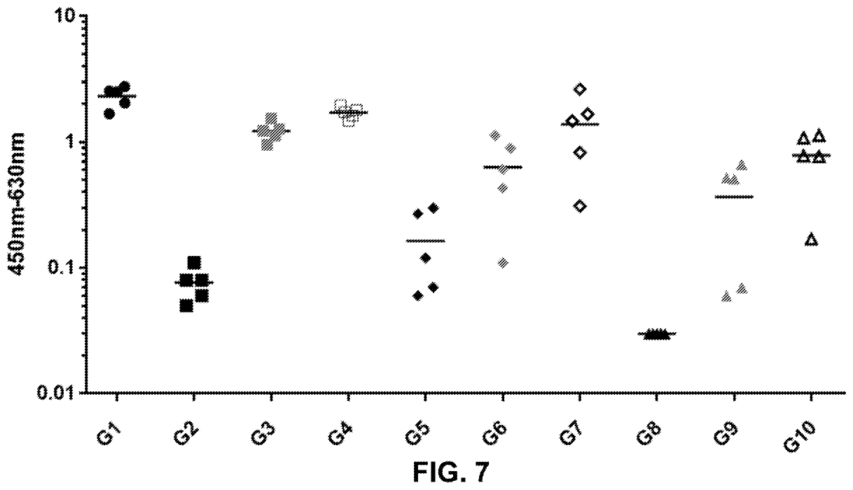

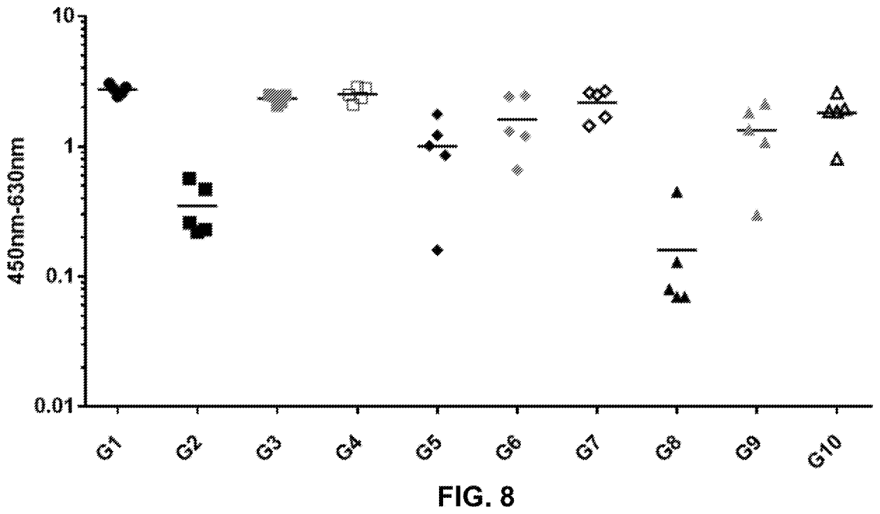

The Pharmacokinetics results show that after injection of different antibodies, the concentration of antibodies in the serum of the hCD40/hFcRn mice decreased with time, which is consistent with pharmacokinetic characteristics. The ELISA results for the serum collected on Day 10, Day 17, and Day 21 are shown in the table below, and FIGS. 6-8, respectively. Overall, the anti-OVA antibodies of all mice in the control and treatment groups showed a continuous increasing trend throughout the experiment. Moreover, the treatment group mice produced less anti-OVA antibodies than the control group mice. The results also showed a dose-correlation, i.e., the higher dose level the antibody was administered, the less anti-OVA antibodies were produced, and the better the immunosuppressive effect was achieved. In general, the immunosuppressive effect of 2F8-IgG1-LALA-YTE was better than that of 12B5-IgG1-LALA-YTE, and the immunosuppressive effect of 12B5-IgG1-LALA-YTE was better than Bleselumab analog.

TABLE 4

| | | Day 10 | Day 17 | Day 21 |
|---|---|---|---|---|
| Group | | (OD value) | (OD value) | (OD value) |
| G1 | MEAN | 0.28 | 2.31 | 2.74 |
| | CV | 0.63 | 0.19 | 0.09 |
| G2 | MEAN | 0.03 | 0.08 | 0.35 |
| | CV | 0.06 | 0.31 | 0.45 |
| G3 | MEAN | 0.03 | 1.22 | 2.34 |
| | CV | 0.05 | 0.17 | 0.09 |
| G4 | MEAN | 0.05 | 1.72 | 2.53 |
| | CV | 0.33 | 0.11 | 0.12 |
| G5 | MEAN | 0.03 | 0.16 | 1.01 |
| | CV | 0.04 | 0.70 | 0.58 |
| G6 | MEAN | 0.03 | 0.64 | 1.61 |
| | CV | 0.08 | 0.62 | 0.50 |
| G7 | MEAN | 0.04 | 1.38 | 2.17 |
| | CV | 0.22 | 0.64 | 0.26 |
| G8 | MEAN | 0.03 | 0.03 | 0.16 |
| | CV | 0.02 | 0.03 | 1.02 |
| G9 | MEAN | 0.03 | 0.37 | 1.34 |
| | CV | 0.06 | 0.77 | 0.53 |
| G10 | MEAN | 0.03 | 0.79 | 1.81 |
| | CV | 0.05 | 0.49 | 0.35 |

CV: coefficient of variation

### Example 5. Pharmacokinetics Analysis

PK Analysis in hCD40 Mice and C57BL/6 Mice

The pharmacokinetic clearance rates of the anti-CD40 antibodies were determined in hCD40 mice. Specifically, the mice were placed into 4 groups (3 mice per group), and administered with 3 mg/kg of Bleselumab analog (G1), 12B5-IgG4-FLAA (G2), 6A4-IgG4-FLAA (G3), and 2F8-IgG4-FLAA (G4), respectively, by intravenous injection. Blood samples were collected 15 minutes, 1 day, 3 days, 7 days, 10 days, and 14 days after administration and 4 days before administration.

The serum levels of human antibodies were determined by sandwich ELISA. Briefly, Goat Anti-Human IgG (H+L) (Jackson ImmunoResearch Inc., Cat #: 109-005-088) was diluted to a final concentration of 2000 ng/ml, added to a 96-well plate (ELISA plate) at 100 μl/well, and then incubated overnight at 4° C. After the incubation, the plate was washed with PBS-T buffer (PBS supplemented with Tween™ 20) 4 times. Antibody-unbound areas were blocked with 2% BSA (bovine serum albumin) for 2 hours at 37° C. Afterwards, the plate was washed with PBS-T buffer 4 times. After washing, 100 μL of serum (1/20 dilution (MRD) in 1% BSA) was added to each well. The wells were sealed and incubated at 37° C. for 1 hour. After washing the plate using a plate washer, Peroxidase AffiniPure F(ab′)₂ Fragment Goat Anti-Human IgG, Fcγ fragment specific (Jackson ImmunoResearch Inc., Cat #: 109-036-098) was added at 100 μl/well to each well of the plate, and incubated at 37° C. for 1 hour. After washing the plate, tetramethylbenzidine (TMB) solution was added at 100 μl/well to the 96-well plate as the substrate. After incubating at room temperature in the dark, 100 μl stop solution (Beyotime, Cat #: P0215) was added to each well. A microplate reader was used to read the absorbance value of each well at wavelengths of 450 nm and 630 nm. Data analysis software Gen5TM was used to analyze the data. The absorbance value and corresponding concentration of the calibration sample prepared by each test product was used to create a standard curve with four parameters (i.e., T_{1/2}, Cₘₐₓ, AUC_{0-14 day}, and CL). The standard curve was used to calculate the antibody concentration of each serum sample. A drug concentration-time curve was created using the calculated sample concentration at each time point. Phoenix™ WinNolin 8.3 was used to calculate the pharmacokinetic parameters. The results are shown in the table below.

Similar to the above experiments, the pharmacokinetic clearance rates of anti-CD40 antibodies Bleselumab analog, 12B5-IgG4-FLAA, 6A4-IgG4-FLAA, and 2F8-IgG4-FLAA were also determined in C57BL/6 mice. Details of the administration scheme are shown in the table below.

TABLE 5

| | | Dosage | | | No. of | Total No. of |
|---|---|---|---|---|---|---|
| Group | Antibodies | (mg/kg) | Route | Mice | mice | administration |
| G1 | Bleselumab analog | 3 | i.v. | hCD40 | 3 | 1 |
| G2 | 12B5-IgG4-FLAA | 3 | i.v. | hCD40 | 3 | 1 |
| G3 | 6A4-IgG4-FLAA | 3 | i.v. | hCD40 | 3 | 1 |
| G4 | 2F8-IgG4-FLAA | 3 | i.v. | hCD40 | 3 | 1 |
| G5 | Bleselumab analog | 3 | i.v. | C57BL/6 | 3 | 1 |
| G6 | 12B5-IgG4-FLAA | 3 | i.v. | C57BL/6 | 3 | 1 |
| G7 | 6A4-IgG4-FLAA | 3 | i.v. | C57BL/6 | 3 | 1 |
| G8 | 2F8-IgG4-FLAA | 3 | i.v. | C57BL/6 | 3 | 1 |

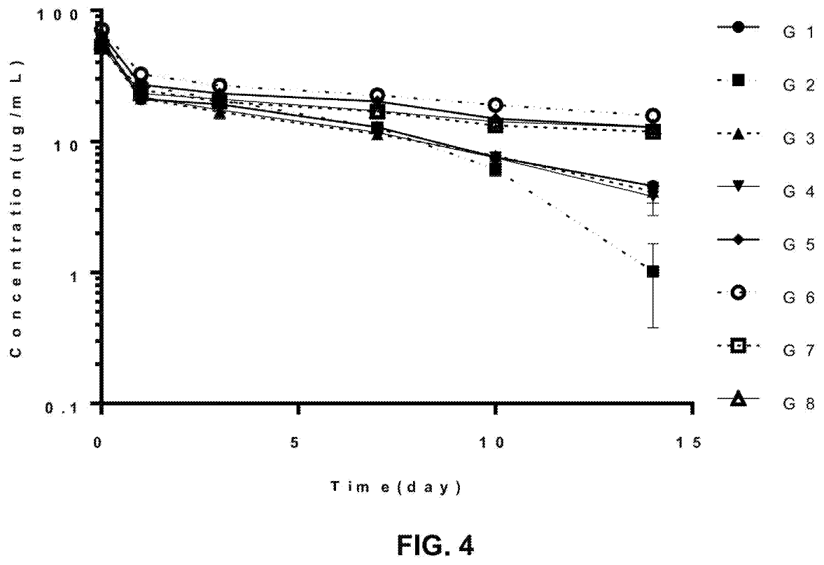

4 days before antibody administration, the antibody concentration was detected as 0 μg/ml (results not shown). As shown in FIG. 4 and the table below, the results were consistent with typical pharmacokinetic characteristics, showing that after injection of different antibodies, the concentration of antibodies in the serum of hCD40 mice and C57BL/6 mice decreased over time. The half-life of anti-CD40 antibodies in C57BL/6 (B6) mice was within the range of 12.37-17.70 days, whereas the half-life of anti-CD40 antibodies in hCD40 mice was within the range of 1.87-4.84 days. The clearance rate (CL) of anti-CD40 antibodies in C57BL/6 mice was in the range of 4.65-6.35 ml/day/kg, whereas the CL of anti-CD40 antibodies in hCD40 mice was in the range of 12.92-14.69 ml/day/kg. The results showed that all antibody concentrations in wild-type C57BL/6 mice were similar. By contrast, in hCD40 mice, all antibody concentrations decreased more rapidly, which may be explained by TMDD (target-mediated drug disposition).

TABLE 6

| | | T_{1/2} | Cₘₐₓ | AUC_{0-14 day} | CL |
|---|---|---|---|---|---|
| Group | | (day) | (μg/ml) | (day*μg/ml) | (ml/day/kg) |
| G1 | MEAN | 4.84 | 60.64 | 200.76 | 12.92 |
| | CV | 0.11 | 0.06 | 0.08 | 0.07 |
| G2 | MEAN | 1.87 | 61.10 | 201.68 | 14.69 |
| | CV | 0.26 | 0.09 | 0.06 | 0.06 |
| G3 | MEAN | 4.77 | 53.65 | 184.74 | 14.11 |
| | CV | 0.12 | 0.04 | 0.06 | 0.09 |
| G4 | MEAN | 4.44 | 53.85 | 187.48 | 14.18 |
| | CV | 0.29 | 0.07 | 0.07 | 0.11 |
| G5 | MEAN | 12.37 | 65.38 | 292.19 | 5.75 |
| | CV | 0.13 | 0.07 | 0.06 | 0.04 |
| G6 | MEAN | 13.49 | 71.48 | 341.94 | 4.65 |
| | CV | 0.19 | 0.04 | 0.04 | 0.11 |
| G7 | MEAN | 12.89 | 52.84 | 253.70 | 6.35 |
| | CV | 0.08 | 0.07 | 0.09 | 0.09 |
| G8 | MEAN | 17.70 | 56.70 | 262.04 | 5.18 |
| | CV | 0.34 | 0.08 | 0.05 | 0.17 |

T_{1/2}: Terminal Half Life
Cₘₐₓ: Max Concentration
AUC_{0-14 day}: Area under Blood Concentration-time Curve 0-14 day
CL: Clearance

PK Analysis in hFcRn Mice

Similar to the above experiments, the pharmacokinetic clearance rate of anti-CD40 antibody 12B5 was determined in hFcRn mice, and the mice were placed into 4 groups (5 mice per group). 2 mg/kg of 12B5-IgG1-LALA-YTE (G1), 12B5-IgG1-N297A-YTE (G2), 12B5-IgG4-YTE (G3), or 12B5-IgG4-FLAA (G4) was administered by intravenous injection. Blood samples were collected 2 hours, 1 day, 3 days, 7 days, 10 days, 14 days, 21 days, 28 days, and 35 days after administration and 4 days before administration. Details of the administration scheme are shown in the table below.

TABLE 7

| | | | | | Total |
|---|---|---|---|---|---|
| | | | | | No. of |
| | | Dosage | | No. of | adminis- |
| Group | Antibodies | (mg/kg) | Route | mice | tration |
| G1 | 12B5-IgG1-LALA-YTE | 2 | i.v. | 5 | 1 |
| G2 | 12B5-IgG1-N297A-YTE | 2 | i.v. | 5 | 1 |
| G3 | 12B5-IgG4-YTE | 2 | i.v. | 5 | 1 |
| G4 | 12B5-IgG4-FLAA | 2 | i.v. | 5 | 1 |

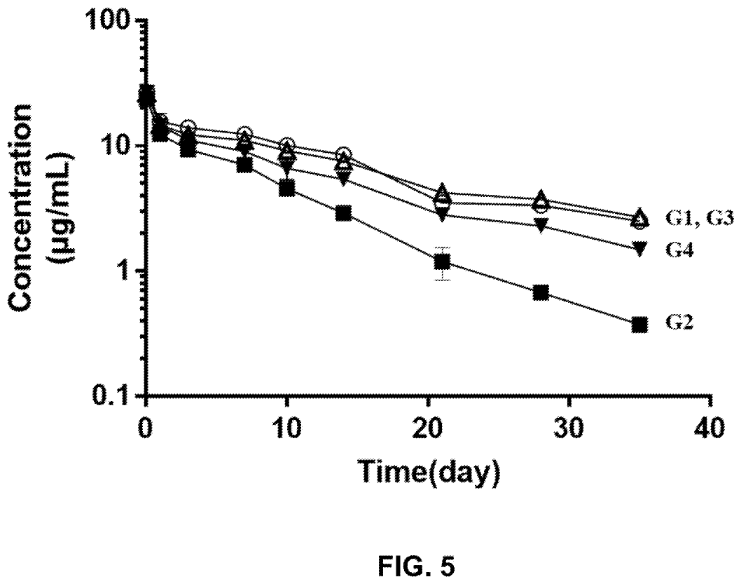

4 days before antibody administration, the antibody concentration was detected as 0 μg/ml (results not shown). As shown in FIG. 5 and the table below, the results were consistent with typical pharmacokinetic characteristics, showing that after injection of different antibodies, the concentration of antibodies in the serum of hFcRn mice decreased over time. The half-life (T_{1/2}) of 12B5-IgG1-LALA-YTE (G1) and 12B5-IgG4-YTE (G3) in mice were 11.99 days and 13.72 days, respectively. 12B5-IgG1-N297A-YTE (G2) exhibited the shortest half-life of 6.58 days. The results showed that 12B5-IgG1-N297A-YTE (G2) had the shortest half-life. The half-life of 12B5-IgG1-LALA-YTE (G1) and 12B5-IgG4-YTE (G3) was longer than that of 12B5-IgG4-FLAA (G4).

TABLE 8

| | | T_{1/2} | Cₘₐₓ | AUC_{0-35 day} | CL |
|---|---|---|---|---|---|
| Group | | (day) | (ug/ml) | (day*ug/ml) | (ml/day/kg) |
| G1 | MEAN | 11.99 | 26.83 | 260.73 | 6.59 |
| | CV | 0.10 | 0.07 | 0.06 | 0.05 |
| G2 | MEAN | 6.58 | 23.76 | 129.08 | 15.09 |
| | CV | 0.04 | 0.09 | 0.03 | 0.03 |
| G3 | MEAN | 13.72 | 26.47 | 249.21 | 6.62 |
| | CV | 0.04 | 0.10 | 0.05 | 0.05 |
| G4 | MEAN | 10.53 | 26.74 | 193.54 | 9.29 |
| | CV | 0.07 | 0.10 | 0.08 | 0.07 |

T_{1/2}: Terminal Half Life;
Cₘₐₓ: Max Concentration;
AUC_{0-35 day}: Area under Blood Concentration-time Curve 0-35 day
CL: Clearance

### Example 6. Binding Affinity of Anti-CD40 Antibodies to Human FcRn

The binding affinity of the anti-CD40 antibodies 12B5-IgG1-LALA-YTE, 12B5-IgG1-N297A-YTE, 12B5-IgG4-YTE and 12B5-IgG4 to human FcRn were measured using surface plasmon resonance (SPR) using Biacore™ (Biacore, Inc., Piscataway N.J.) 8K or T200 biosensor equipped with pre-immobilized Protein A sensor chips.

hFcRn-His (FCGRT&B2M Heterodimer Protein, His Tag&Strep II Tag (SPR & BLI & MALS verified, Beijing ACROBiosystems Co. Ltd., Cat #: FCM-H5286)) was diluted to 1 μg/ml with 1×HBS-EP+ buffer (PH 7.4) and then injected into the Biacore™ 8K or T200 biosensor at 10 μl/min for about 50 seconds to meet the required capture level (e.g., about 200 response units (RU)). Purified anti-CD40 antibodies at concentrations of 1250 nM, 625 nM, 312.5 nM, 156.25 nM, 78.125 nM or 0 nM (with 1×HBS-EP+ buffer (PH 7.4)) were then injected at 30 μl/min for 50 seconds. Dissociation was monitored for 200 seconds. The chip was regenerated after the last injection of each titration with a glycine solution (pH 1.7) at 30 μl/min for 30 seconds. The binding curve was obtained. Data analysis was performed by analysis software, and the association and dissociation curves were fitted using the Steady State Affinity binding model to obtain affinity kinetic data.

The above experiment was repeated except that the 1×HBS-EP+ buffer (pH 7.4) was replaced with 1×HBS-EP+ buffer (pH 6.0).

As a person of ordinary skill in the art would understand, the same method with appropriate adjustments for parameters (e.g., antibody concentration) was performed for each tested antibody. The results for the tested antibodies are summarized in the table below.

The results are shown in the table below. When the pH value was 7.4, 12B5-IgG4-FLAA did not bind to hFcRn, while other antibodies bound to hFcRn. At pH 6.0, all antibodies exhibited high binding affinity to hFcRn. The results indicate that under acidic conditions, all antibodies had increased affinity for hFcRn. In addition, 12B5-IgG1-LALA-YTE and 12B5-IgG4-YTE showed higher binding affinity to hFcRn than 12B5-IgG1-N297A-YTE.

TABLE 9

| Capture 1 Solution | Analyte 1 Solution | KD (M) |
|---|---|---|
| 12B5-IgG1-LALA-YTE | hFcRn-His (PH 6.0) | 3.78E−08 |
| 12B5-IgG1-N297A-YTE | hFcRn-His (PH 6.0) | 1.12E−07 |
| 12B5-IgG4-YTE | hFcRn-His (PH 6.0) | 9.10E−08 |
| 12B5-IgG4-FLAA | hFcRn-His (PH 6.0) | 4.97E−07 |
| 12B5-IgG1-LALA-YTE | hFcRn-His (PH 7.4) | 1.18E−06 |
| 12B5-IgG1-N297A-YTE | hFcRn-His (PH 7.4) | 2.68E−06 |
| 12B5-IgG4-YTE | hFcRn-His (PH 7.4) | 2.06E−06 |
| 12B5-IgG4-FLAA | hFcRn-His (PH 7.4) | — |

### Example 7. Determination of Antibody-Dependent Cellular Cytotoxicity (ADCC)

Rituximab (VH SEQ ID NO: 67, VL SEQ ID NO: 68) is a chimeric monoclonal antibody targeting CD20, and was first launched in 1997 as an intravenous treatment for relapsed or refractory low-grade or follicular, CD20-positive B-cell non-Hodgkin lymphoma (NHL).

In this experiment, the FcR-TANK cells (ImmuneOnco Biopharmaceuticals (Shanghai) Inc.) were used as effector cells, and Raji cells (ATCC, Cat #: CCL-86) were used as target cells. The cells were incubated with each antibody (final concentration 50 μg/mL) at an E:T (effector: target) ratio of 3:1 for 2 hours or 4 hours to determine ADCC activity.

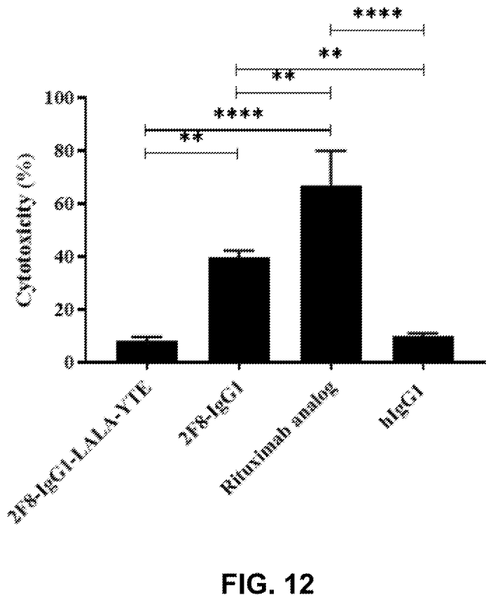

The results are showed in FIG. 12. 2F8-IgG1 and Rituximab analog have ADCC activity, but 2F8-IgG1-LALA-YTE showed no ADCC effect.

### Example 8. Determination of Complement Dependent Cytotoxicity (CDC)

Experiments were performed to evaluate CDC effects of anti-CD40 antibodies. In the experiment, Raji cells (cell density 5×10⁴ cells/well) were seeded in a 96-well plate, and then the antibody 2F8-IgG1-LALA-YTE, Rituximab analog, or human IgG1 at a final concentration of 50 μg/mL was added. The cells were incubated with each antibody at 37° C. for 30 minutes, and then diluted Normal Human Serum Complement (QIDEL, Cat #: A113) was added. After incubating at 37° C. 5% CO₂ for 1.5 hours, PrestoBlue™ Cell Viability Reagent (Invitrogen, Cat #: 2413466) was added to each well, and the plates were incubated at 37° C. for 2 hours with 5% CO₂.

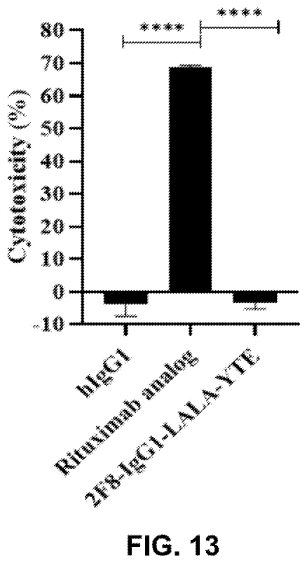

The results are shown in FIG. 13. 2F8-IgG1-LALA-YTE exhibited no CDC effect, while the positive control Rituximab analog exhibited strong CDC effect.

### Example 9. Inhibitory Effect of Anti-CD40 Antibody on PBMC Proliferation

Peripheral blood mononuclear cell (PBMC) cells were seeded in a 96-well plate (5×10⁴ cells/well). Serially diluted antibody 2F8-IgG1-LALA-YTE, or hIgG1 (100 ng/mL, 10 ng/mL, 8 ng/ml, 5 ng/mL, 4 ng/mL, 2 ng/mL, 1 ng/mL, 0.5 ng/ml or 0.25 ng/mL) was added to each well and incubated at 37° C. for 1 hour. Then 10 μl ANTI-FLAG antibody (Sigma, Cat #: F1804) conjugated shCD154 (Human CD40 Ligand/TNFSF5 Protein, His, Flag Tag (active trimer MALS verified), ACRO, Cat #: CDL-H52Db) was added to each well, and the plates were incubated at 37° C. for 64 hours. The fluorescence signal was measured using the Vazyme Biotech CellCounting-Lite® 2.0 Luminescent Cell Viability Assay (Vazyme, Cat #: DD1101-02) via a microplate reader.

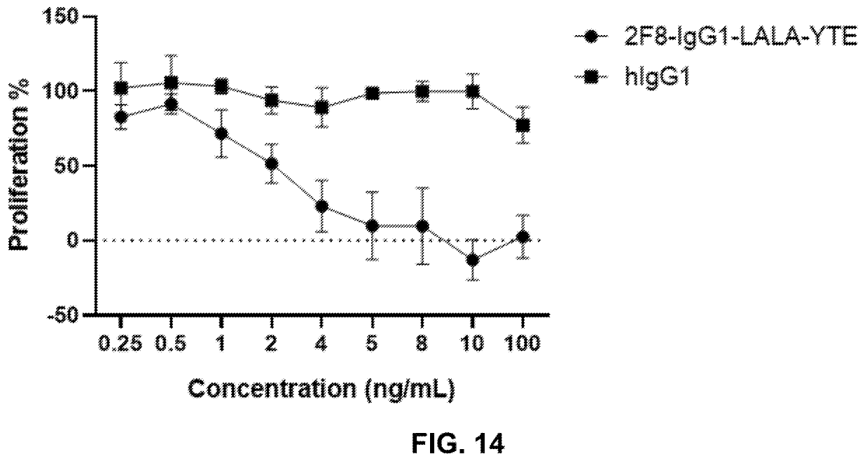

The results are shown in FIG. 14, which indicated that 2F8-IgG1-LALA-YTE inhibited PBMC proliferation effectively.

### Example 10. Inhibitory Effect of Anti-CD40 Antibody on B Cells Proliferation

In the peripheral blood, B cell subsets can be distinguished corresponding to different stages of differentiation, maturation, and activation, which are characterized by the expression of different surface markers, such as CD19, CD20, CD27, CD23, CD69, CD80 and CD86, wherein CD23, CD69, CD80 and CD86 are special surface markers for active B cells.

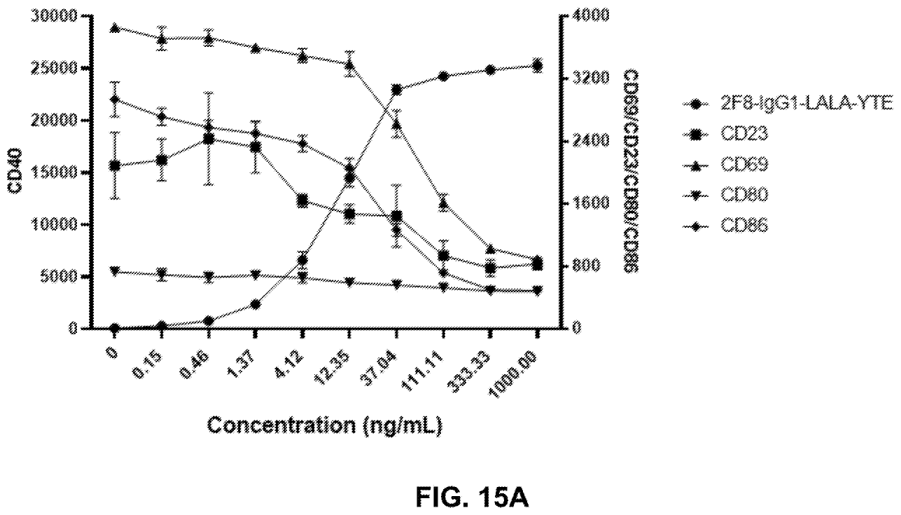

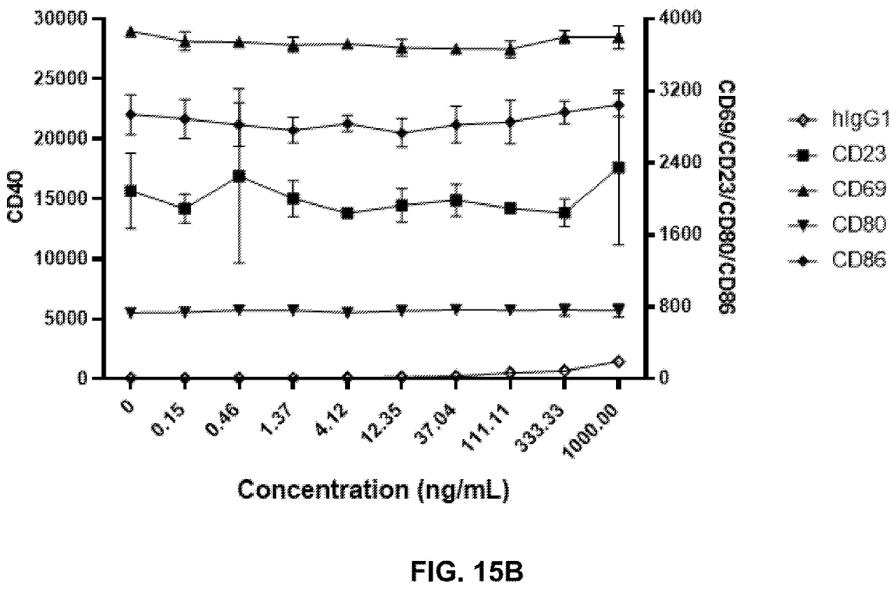

In this experiment, human PBMC cells were plated in a 96-well plate at a density of 1×10⁵ cells/well. Anti-CD40 antibody 2F8-IgG1-LALA-YTE and hIgG1 were serially diluted (3-fold) with the highest concentration of 1000 ng/mL. 10 μL antibody was added to the 96-well plate. After incubating at 37° C. for 1 hour, 10 μL Anti-FLAG® M2 antibody (Sigma, Cat #: F1804) conjugated shCD154 was added and incubated at 37° C. for 18 hours. Then, the cells were incubated with PE anti-human CD20 (BioLegend, Cat #: 302306), PerCP/Cyanine5.5 anti-human CD69 Antibody (BioLegend, Cat #: 310926), Brilliant Violet 421™ anti-human CD86 Antibody (BioLegend, Cat #: 305426), FITC anti-human CD80 Antibody (BioLegend, Cat #: 375406), APC/Cy7 anti-human CD23 (BioLegend, Cat #: 338502), Human CD40 Ligand/TNFSF5 Protein, His, Flag Tag (active trimer) (MALS verified) (ACRO, Cat #: CDL-H52Db-100 ug), and Monoclonal ANTI-FLAG® M2 antibody (Sigma, Cat #: F1804-1 MG) at 4° C. in the dark for 30 minutes before flow cytometry analysis. The results are shown in FIGS. 15A-15B.

With the increase of the concentration of 2F8-IgG1-LALA-YTE, the percentage of activated B cell subsets all decreased, indicating that 2F8-IgG1-LALA-YTE effectively inhibited the proliferation and activation of B cells.

### Example 11. Evaluation Effects of Anti-CD40 Antibodies Using an EAE Model of hCD40/hFcRn Mice

Experimental autoimmune encephalomyelitis (EAE) is the most commonly used experimental model for the human inflammatory demyelinating disease, multiple sclerosis (MS). In this experiment, EAE model was established by hCD40/hFcRn mice to analyze the effects of the human anti-CD40 antibodies. Specifically, hCD40/hFcRn mice were placed into a control group (G1) and two treatment groups (G2 and G3). Then the hCD40/hFcRn mice were immunized by injecting 100 μL MOG (Myelin Oligodendrocyte Glycoprotein (35-55)) (at a dose level of 300 μg/mouse) into the posterior ribs at two points on Day 1 (one day after grouping), and 250 μL PTX (Pertussis Toxin) was injected intraperitoneally at 2 and 48 hours after immunization to obtain an EAE model. The control group (G1) mice were injected with an equal volume of PBS.

G2 and G3 group mice were administered with 10 mg/kg of hIgG1 or 2F8-IgG1-LALA-YTE, respectively, by intraperitoneally injection on Day 0 (the day of grouping), Day 4 (4 days after grouping), and Day 8 (8 days after grouping). The control group mice (G1) were not injected. Mice were weighed and examined daily for the neurological symptoms of EAE, scored according to the following scale: 0=no symptoms; 1=floppy tail; 2=hindlimb weakness; 3=hindlimb paralysis; 4=forelimb paralysis; 5=mouse moribund or dead; and 0.5 gradations represent intermediate scores. At the end of the experiment (30 days after grouping), spinal cords were collected for histopathological analysis. Details of the administration scheme were shown in the table below.

TABLE 10

| | | | | | Total No. of |
|---|---|---|---|---|---|
| | | Dosage | | No. of | adminis- |
| Group | Antibodies | (mg/kg) | Route | mice | tration |
| G1 | — | — | — | 8 | — |
| G2 | hIgG1 | 10 | i.p. | 8 | 3 |
| G3 | 2F8-IgG1-LALA-YTE | 10 | i.p. | 8 | 3 |

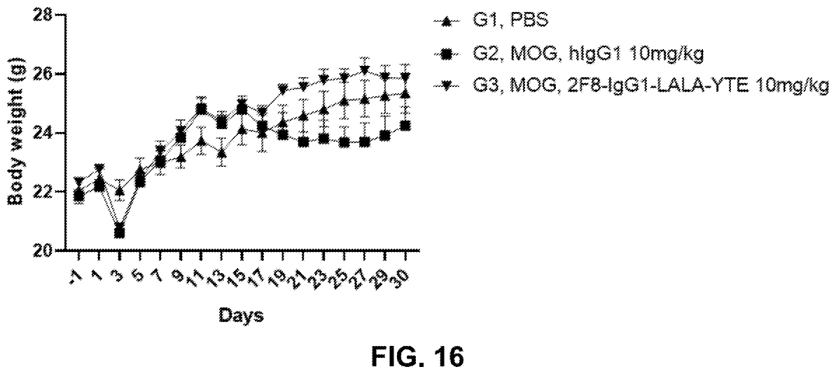

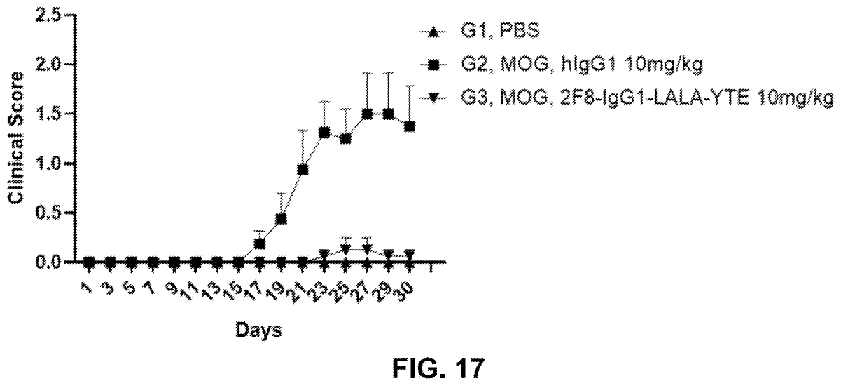

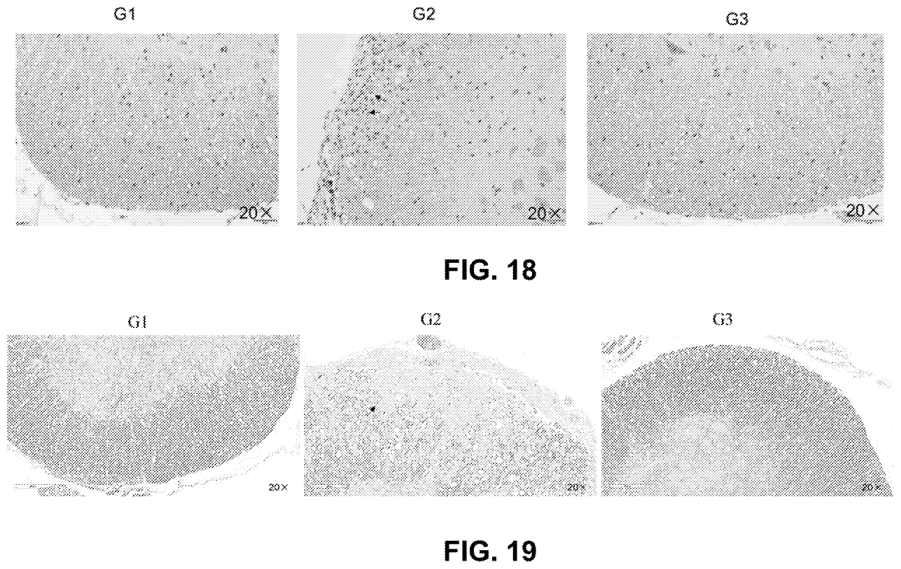

As shown in FIGS. 16-19, 2F8-IgG1-LALA-YTE (G3) could prevent weight loss caused by animal modeling (FIG. 16), and reduce clinical scores (FIG. 17). There was also had a certain improvement effect on the infiltration of myelitis cells (FIG. 18) and spinal cord demyelination (FIG. 19) in EAE mice administered with 2F8-IgG1-LALA-YTE.

### Example 12. Evaluation Effects of Anti-CD40 Antibodies Using a CIA Model of hCD40 Mice

Collagen Induced Arthritis (CIA) model is a commonly used model as it shares immunological and pathological similarities to human rheumatoid arthritis (RA). CIA model was established by hCD40 mice to analyze the effects of the human anti-CD40 antibodies.

Specifically, hCD40 mice were placed into a control group (G1) and two model groups (G2 and G3). Then the hCD40 mice were immunized by injecting 50 μL Type II collagen (CII) emulsion (consisting of equal volumes of CFA and Collagen from chicken sternal cartilage (SIGMA Cat #: C9301)) into the root of tail at two points on Day 0 (the first day of immunization). The second immunization was performed on Day 21 (21 days after the first immunization) with the same dose to obtain CIA models. The control group (G1) were injected with an equal volume of PBS.

G3 mice were administered with 3 mg/kg 12B5-IgG1-LALA-YTE by intraperitoneal injection on Day 0, Day 4, Day 7, Day 20, Day 24, and Day 27. G2 mice were administered with an equal volume of PBS. G1 mice were not injected. All the mice were weighed and were monitored daily for signs of erythema and swelling of: (A) the interphalangeal joints of the digits, (B) the metacarpophalangeal joints and (C) wrist in the forepaws and the metatarsophalangeal joints and ankle joints in the hindpaws. Severity of clinical arthritis in individual paws was scored on a scale of 0-4 as follows:
0=Normal. No obvious differences in appearance vs. healthy mice;
1=One type of joint (A, B, or C) erythema and swollen;
2=Two types of joint (A, B, or C) erythema and swollen;
3=All three types of joint (A, B, or C) erythema and swollen; and
4=Entire paw erythema and swollen.

The animal's score is the sum of all four limbs scores on scale of 0-16. At the end of the experiment (56 days after the first immunization), the limbs were collected for histopathological analysis. Details of the administration scheme were shown in the table below.

TABLE 11

| | | | | | Total |
|---|---|---|---|---|---|
| | | | | | No. of |
| | | Dosage | | No. of | adminis- |
| Group | Antibody | (mg/kg) | Route | mice | tration |
| G1 | — | — | — | 10 | — |
| G2 | PBS | — | i.p. | 10 | 6 |
| G3 | 12B5-IgG1-LALA-YTE | 3 | i.p. | 10 | 6 |

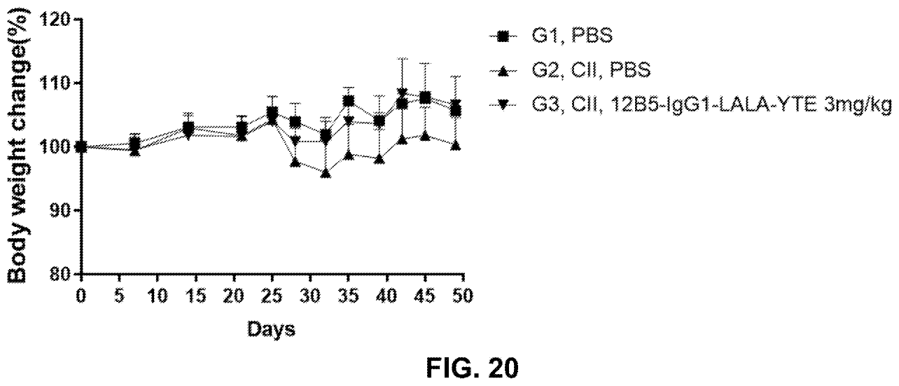

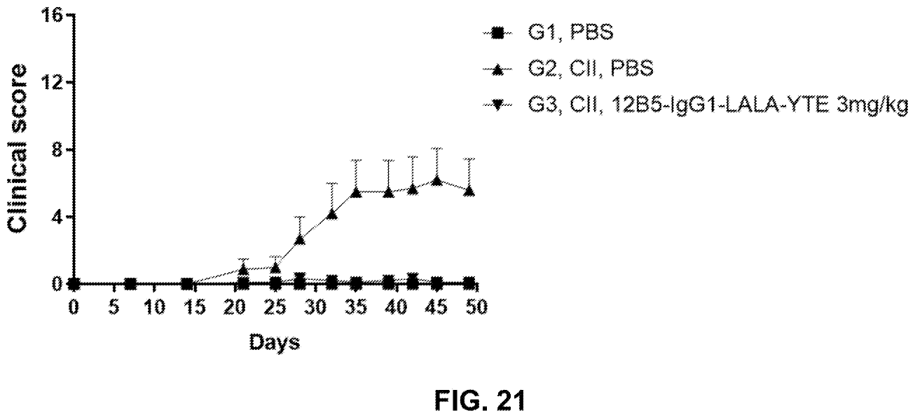

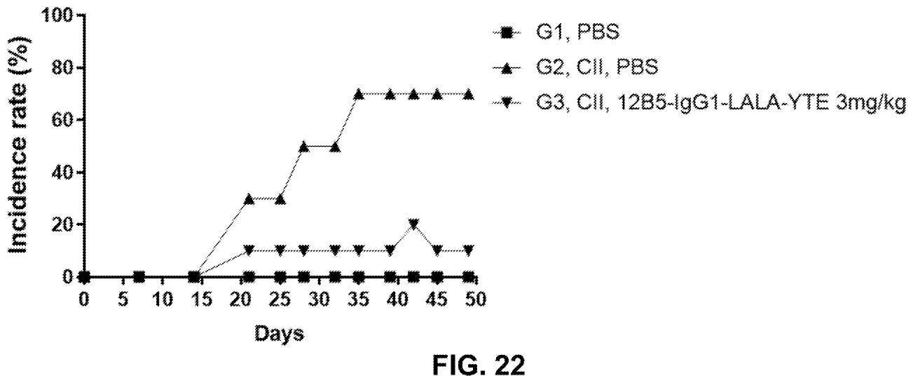

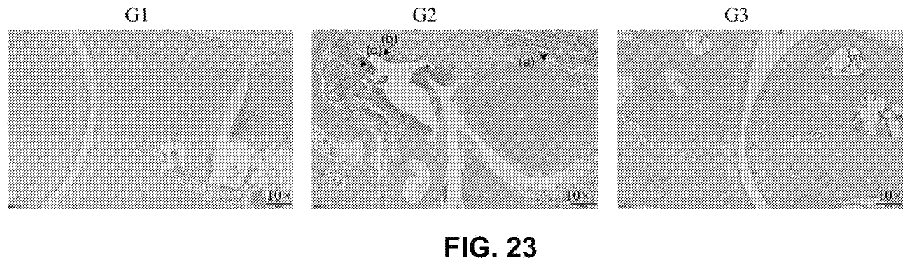

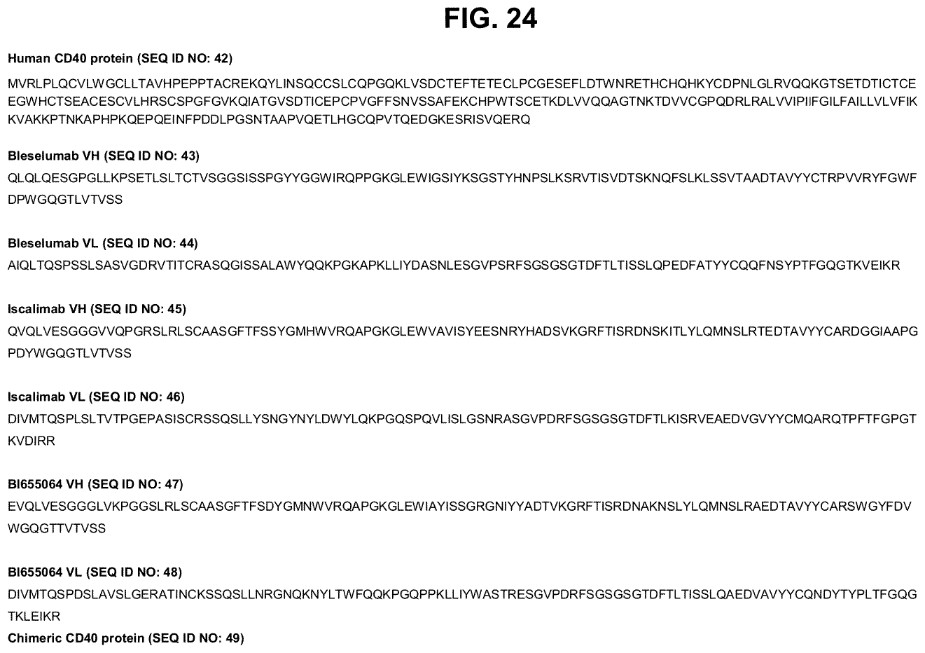

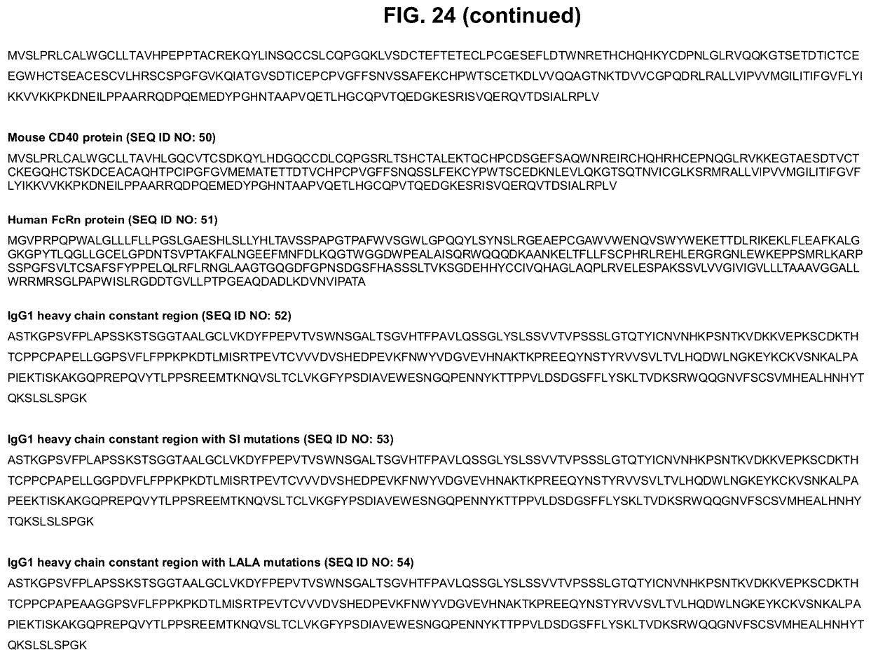

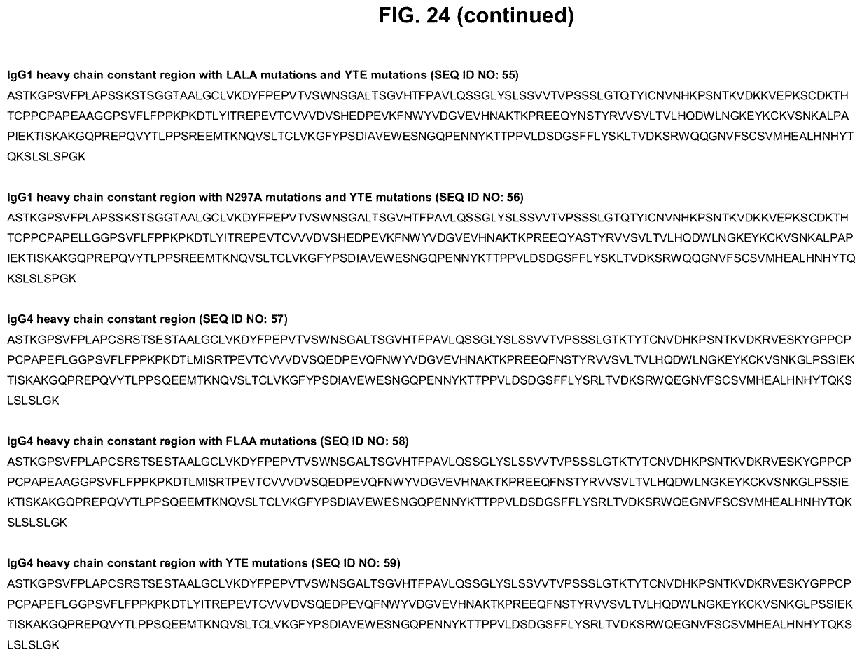

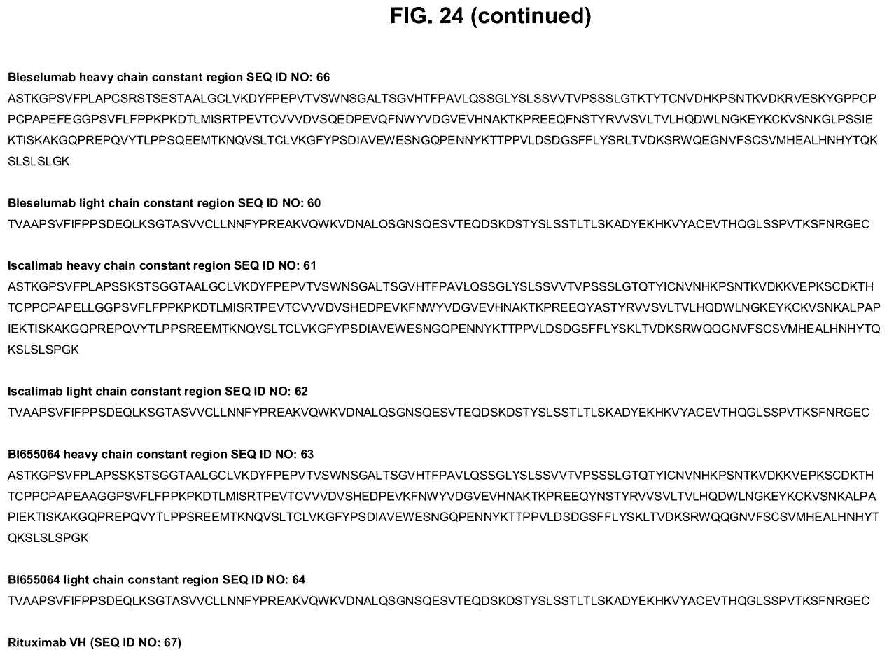

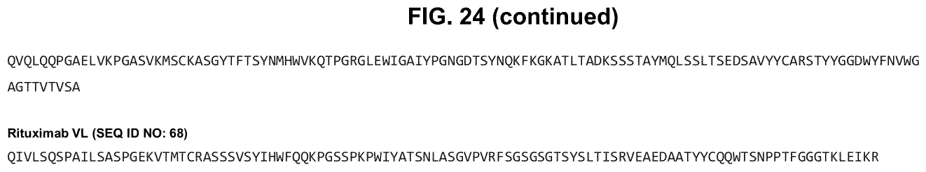

As shown in FIGS. 20-23, 12B5-IgG1-LALA-YTE (G3) could prevent weight loss caused by animal modeling (FIG. 20), and significantly reduce the clinical severity, as revealed by lower clinical scores and incidence rates as compared to control group G2 (FIGS. 21-22). As shown in FIG. 23, the histopathological analysis results of joint tissues demonstrated that inflammatory cell influx (a), synovial hyperplasia (b), pannus formation (c), and spinal cord demyelination were also ameliorated in 12B5-IgG1-LALA-YTE treated group versus controls.

### Example 13. In Vivo Efficacy of Anti-CD40 Antibody

Allergic Dermatitis (AD) is an inflammatory condition of the skin characterized by erythema, pruritus, scaling, lichenification, and papulovesicles.

In this experiment, the in vivo efficacy of anti-CD40 antibody 2F8-IgG1-LALA-YTE in a dog diagnosed with AD was evaluated. Prior to the experiment, the dog had recurrent AD symptoms for over 1 year and received repeated glucocorticoid treatment. Specifically, the antibody was administered by intravenous (i.v.) injection. The injection volume was calculated based on the weight of the dog. The frequency of administration was once every 2 weeks (6 administrations in total). After two injections, it was observed that erythema reduced significantly on the dog's skin. Peripheral blood was also collected to test the blood biochemistry and complete blood count (CBC). The results showed that the tested antibody 2F8-IgG1-LALA-YTE was well tolerated and not toxic to the dog. No adverse reactions were observed.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to CD40 (TNF Receptor Superfamily Member 5) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(4) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(5) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 28, 29, 30, respectively; and
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively.

2. The antibody or antigen-binding fragment thereof of claim 1, wherein the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 4, 5, and 6, respectively.

3. The antibody or antigen-binding fragment thereof of claim 1, wherein the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively.

4. The antibody or antigen-binding fragment thereof of claim 1, wherein the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively.

5. The antibody or antigen-binding fragment thereof of claim 1, wherein the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 22, 23, and 24, respectively.

6. The antibody or antigen-binding fragment thereof of claim 1, wherein the VH comprises CDRs 1, 2, 3 with the amino acid sequences set forth in SEQ ID NOs: 25, 26 and 27, respectively, and the VL comprises CDRs 1, 2, 3 with the amino acid sequences set forth in SEQ ID NOs: 28, 29, and 30, respectively.

7. The antibody or antigen-binding fragment thereof of claim 1, wherein the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 34, 35, and 36, respectively.

8. The antibody or antigen-binding fragment thereof of any one of claims 1-7, wherein the antibody or antigen-binding fragment thereof specifically binds to human, monkey, or dog CD40.

9. The antibody or antigen-binding fragment thereof of any one of claims 1-8, wherein the antibody or antigen-binding fragment thereof is a human antibody or antigen-binding fragment thereof, a single-chain variable fragment (scFv), and/or a multi-specific antibody (e.g., a bispecific antibody).

10. The antibody or antigen-binding fragment thereof of any one of claims 1-9, wherein the antibody or antigen-binding fragment thereof is a human IgG1 antibody or antigen-binding fragment thereof (optionally with YTE and/or LALA mutations) or a human IgG4 antibody or antigen-binding fragment thereof (optionally with YTE).

11. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 37 binds to CD40;
(2) an immunoglobulin light chain or a fragment thereof comprising a VL comprising complementarity determining regions (CDRs) 1, 2, and 3 comprising the amino acid sequences set forth in SEQ ID NOs: 4, 5, and 6, respectively, and wherein the VL, when paired with a VH comprising the amino acid sequence set forth in SEQ ID NO: 65 binds to CD40;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 39 binds to CD40;
(4) an immunoglobulin light chain or a fragment thereof comprising a VL comprising complementarity determining regions (CDRs) 1, 2, and 3 comprising the amino acid sequences set forth in SEQ ID NOs: 10, 11, and 12, respectively, and wherein the VL, when paired with a VH comprising the amino acid sequence set forth in SEQ ID NO: 38 binds to CD40;
(5) an immunoglobulin heavy chain or a fragment thereof comprising a VH comprising complementarity determining regions (CDRs) 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 41 binds to CD40; or
(6) an immunoglobulin light chain or a fragment thereof comprising a VL comprising CDRs 1, 2, and 3 comprising the amino acid sequences set forth in SEQ ID NOs: 16, 17 and 18, respectively, and wherein the VL, when paired with a VH comprising the amino acid sequence set forth in SEQ ID NO: 40 binds to CD40.

12. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively.

13. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively.

14. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOS: 7, 8, and 9, respectively.

15. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively.

16. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively.

17. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

18. The nucleic acid of any one of claims 11-17, wherein the VH when paired with a VL specifically binds to human, monkey, or dog CD40, or the VL when paired with a VH specifically binds to human, monkey, or dog CD40.

19. The nucleic acid of any one of claims 11-18, wherein the immunoglobulin heavy chain or the fragment thereof is a human immunoglobulin heavy chain or a fragment thereof (e.g., a human IgG1 heavy chain or a fragment thereof, optionally with YTE and/or LALA mutations; or a human IgG4 heavy chain or a fragment thereof, optionally with YTE), and the immunoglobulin light chain or the fragment thereof is a human immunoglobulin light chain or a fragment thereof.

20. The nucleic acid of any one of claims 11-19, wherein the nucleic acid encodes a single-chain variable fragment (scFv) or a multi-specific antibody (e.g., a bispecific antibody).

21. The nucleic acid of any one of claims 11-20, wherein the nucleic acid is cDNA.

22. A vector comprising one or more of the nucleic acids of any one of claims 11-21.

23. A vector comprising two of the nucleic acids of any one of claims 11-21, wherein the vector encodes the VL region and the VH region that together bind to CD40.

24. A pair of vectors, wherein each vector comprises one of the nucleic acids of any one of claims 11-21, wherein together the pair of vectors encodes the VL region and the VH region that together bind to CD40.

25. A cell comprising the vector of claim 22 or 23, or the pair of vectors of claim 24.

26. The cell of claim 25, wherein the cell is a CHO cell.

27. A cell comprising one or more of the nucleic acids of any one of claims 11-21.

28. A cell comprising two of the nucleic acids of any one of claims 11-21.

29. The cell of claim 28, wherein the two nucleic acids together encode the VL region and the VH region that together bind to CD40.

30. A method of producing an antibody or an antigen-binding fragment thereof, the method comprising
(a) culturing the cell of any one of claims 25-29 under conditions sufficient for the cell to produce the antibody or the antigen-binding fragment; and
(b) collecting the antibody or the antigen-binding fragment produced by the cell.

31. An antibody or antigen-binding fragment thereof that binds to CD40 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 65, and the selected VL sequence is SEQ ID NO: 37;
(2) the selected VH sequence is SEQ ID NO: 38, and the selected VL sequence is SEQ ID NO: 39; and
(3) the selected VH sequence is SEQ ID NO: 40, and the selected VL sequence is SEQ ID NO: 41.

32. The antibody or antigen-binding fragment thereof of claim 31, wherein the VH comprises the sequence of SEQ ID NO: 65 and the VL comprises the sequence of SEQ ID NO: 37.

33. The antibody or antigen-binding fragment thereof of claim 31, wherein the VH comprises the sequence of SEQ ID NO: 38 and the VL comprises the sequence of SEQ ID NO: 39.

34. The antibody or antigen-binding fragment thereof of claim 31, wherein the VH comprises the sequence of SEQ ID NO: 40 and the VL comprises the sequence of SEQ ID NO: 41.

35. The antibody or antigen-binding fragment thereof of any one of claims 31-34, wherein the antibody or antigen-binding fragment thereof specifically binds to human, monkey, or dog CD40.

36. The antibody or antigen-binding fragment thereof of any one of claims 31-35, wherein the antibody or antigen-binding fragment thereof is a human antibody or antigen-binding fragment thereof, a single-chain variable fragment (scFv), and/or a multi-specific antibody (e.g., a bispecific antibody).

37. The antibody or antigen-binding fragment thereof of any one of claims 31-36, wherein the antibody or antigen-binding fragment is a human IgG1 antibody or antigen-binding fragment thereof (optionally with YTE and/or LALA mutations) or a human IgG4 antibody or antigen-binding fragment thereof (optionally with YTE).

38. An antibody or antigen-binding fragment thereof comprising the VH CDRs 1, 2, 3, and the VL CDRs 1, 2, 3 of the antibody or antigen-binding fragment thereof of any one of claims 1-10 and 31-37.

39. An antibody or antigen-binding fragment thereof that cross-competes with the antibody or antigen-binding fragment thereof of any one of claims 1-10 and 31-38.

40. An antibody-drug conjugate comprising the antibody or antigen-binding fragment thereof of any one of claims 1-10 and 31-39 covalently bound to a therapeutic agent.

41. The antibody drug conjugate of claim 40, wherein the therapeutic agent is a cytotoxic or cytostatic agent.

42. A method of treating a subject having cancer, the method comprising administering a therapeutically effective amount of a composition comprising the antibody or antigen-binding fragment thereof of any one of claims 1-10 and 31-39, or the antibody-drug conjugate of claim 40 or 41, to the subject.

43. The method of claim 42, wherein the subject has a solid tumor.

44. The method of claim 42, wherein the cancer is melanoma, pancreatic carcinoma, mesothelioma, or a hematological malignancy.

45. The method of claim 42, wherein the cancer is Non-Hodgkin's lymphoma, lymphoma, or chronic lymphocytic leukemia.

46. A method of decreasing the rate of tumor growth, the method comprising
contacting a tumor cell with an effective amount of a composition comprising an antibody or antigen-binding fragment thereof of any one of claims 1-10 and 31-39, or the antibody-drug conjugate of claim 40 or 41.

47. A method of killing a tumor cell, the method comprising
contacting a tumor cell with an effective amount of a composition comprising the antibody or antigen-binding fragment thereof of any one of claims 1-10 and 31-39, or the antibody-drug conjugate of claim 40 or 41.

48. A method of inhibiting immune response in a subject, the method comprising
administering to the subject an effective amount of a composition comprising the antibody or antigen-binding fragment thereof of any one of claims 1-10 and 31-39, or the antibody-drug conjugate of claim 40 or 41.

49. The method of claim 48, wherein the subject has an autoimmune disease.

50. A method of treating an autoimmune disease, the method comprising
administering to the subject an effective amount of a composition comprising the antibody or antigen-binding fragment thereof of any one of claims 1-10 and 31-39, or the antibody-drug conjugate of claim 40 or 41.

51. The method of claim 50, wherein the autoimmune disease is rheumatoid arthritis, systemic lupus erythematosus, lupus nephritis, allergic dermatitis, or multiple sclerosis.

52. A pharmaceutical composition comprising the antibody or antigen-binding fragment thereof of any one of claims 1-10 and 31-39, and a pharmaceutically acceptable carrier.

53. A pharmaceutical composition comprising the antibody drug conjugate of claim 40 or 41, and a pharmaceutically acceptable carrier.

54. An antibody or antigen-binding fragment thereof that binds to CD40 (TNF Receptor Superfamily Member 5) comprising a Fc region, wherein the Fc region lacks ADCC effect or has reduced ADCC effect as compared to a wild-type Fc region.

55. The antibody or antigen-binding fragment thereof of claim 54, wherein the KD between the antibody or antigen-binding fragment thereof and FcRn (e.g., human FcRn) is less than 1×10⁻⁵ M, less than 5×10⁻⁶ M, less than 1×10⁻⁶ M, less than 5×10⁻⁷ M, less than 1×10⁻⁷ M, or less than 5×10⁻⁸ M.

56. The antibody or antigen-binding fragment thereof of claim 54 or 55, wherein the Fc region is IgG1 or IgG4 subtype.

57. The antibody or antigen-binding fragment thereof of any one of claims 54-56, wherein the Fc region comprises YTE mutations.

58. The antibody or antigen-binding fragment thereof of any one of claim 54, 55 and 57, wherein the Fc region comprises LALA mutations.

59. The antibody or antigen-binding fragment thereof of any one of claims 1-10, 31-37, and 54-58, wherein the half-life of the antibody or antigen-binding fragment thereof is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days when administered to a subject.

60. The antibody or antigen-binding fragment thereof of claim 59, wherein the subject is a mouse. The antibody or antigen-binding fragment thereof of claim 59 or 60, wherein the subject is genetically-modified to express a human or humanized CD40.


SEQUENCE LISTING (68 sequences)

SEQ ID NO: 1 - protein, 5 aa, synthetic construct
SYAMH

SEQ ID NO: 2 - protein, 17 aa, synthetic construct
VISYDGDNKYHAESVKG

SEQ ID NO: 3 - protein, 15 aa, synthetic construct
RGTVTNYYSYSGLDV

SEQ ID NO: 4 - protein, 16 aa, synthetic construct
RSSQSLLYSNGKNYLD

SEQ ID NO: 5 - protein, 7 aa, synthetic construct
LASNRAS

SEQ ID NO: 6 - protein, 9 aa, synthetic construct
MQALQTPRT

SEQ ID NO: 7 - protein, 5 aa, synthetic construct
NYVMS

SEQ ID NO: 8 - protein, 17 aa, synthetic construct
TIRGSGISTYYTDSVKG

SEQ ID NO: 9 - protein, 11 aa, synthetic construct
GIVGATTPFDY

SEQ ID NO: 10 - protein, 11 aa, synthetic construct
RASQSVRSFLA

SEQ ID NO: 11 - protein, 7 aa, synthetic construct
ETSNRAT

SEQ ID NO: 12 - protein, 9 aa, synthetic construct
QQRSDWPPT

SEQ ID NO: 13 - protein, 5 aa, synthetic construct
YYYWS

SEQ ID NO: 14 - protein, 16 aa, synthetic construct
HISYSGSTNYNPSLKS

SEQ ID NO: 15 - protein, 11 aa, synthetic construct
GIVGARRGFDF

SEQ ID NO: 16 - protein, 11 aa, synthetic construct
RASQSVSNNLA

SEQ ID NO: 17 - protein, 7 aa, synthetic construct
DASNRAA

SEQ ID NO: 18 - protein, 9 aa, synthetic construct
QQRNNWPPT

SEQ ID NO: 19 - protein, 7 aa, synthetic construct
GFSFSSY

SEQ ID NO: 20 - protein, 6 aa, synthetic construct
SYDGDN

SEQ ID NO: 21 - protein, 15 aa, synthetic construct
RGTVTNYYSYSGLDV

SEQ ID NO: 22 - protein, 16 aa, synthetic construct
RSSQSLLYSNGKNYLD

SEQ ID NO: 23 - protein, 7 aa, synthetic construct
LASNRAS

SEQ ID NO: 24 - protein, 9 aa, synthetic construct
MQALQTPRT

SEQ ID NO: 25 - protein, 7 aa, synthetic construct
GFTFSNY

SEQ ID NO: 26 - protein, 6 aa, synthetic construct
RGSGIS

SEQ ID NO: 27 - protein, 11 aa, synthetic construct
GIVGATTPFDY

SEQ ID NO: 28 - protein, 11 aa, synthetic construct
RASQSVRSFLA

SEQ ID NO: 29 - protein, 7 aa, synthetic construct
ETSNRAT

SEQ ID NO: 30 - protein, 9 aa, synthetic construct
QQRSDWPPT

SEQ ID NO: 31 - protein, 7 aa, synthetic construct
GGSISYY

SEQ ID NO: 32 - protein, 5 aa, synthetic construct
SYSGS

SEQ ID NO: 33 - protein, 11 aa, synthetic construct
GIVGARRGFDF

SEQ ID NO: 34 - protein, 11 aa, synthetic construct
RASQSVSNNLA

SEQ ID NO: 35 - protein, 7 aa, synthetic construct
DASNRAA

SEQ ID NO: 36 - protein, 9 aa, synthetic construct
QQRNNWPPT

SEQ ID NO: 37 - protein, 113 aa, synthetic construct
DIQLTQSPLSLPVTPGEPASISCRSSQSLLYSNGKNYLDWYLQKPGQSPQLLIYLASNRASGVPDRFSGSGSGTDFTLKISRVEAEDVGVYFCMQALQTPRTFGQGTKVEIKR

SEQ ID NO: 38 - protein, 120 aa, synthetic construct
QVQLVQSGGGLVQPGGSLRLSCAASGFTFSNYVMSWVRQAPGKGLEWVSTIRGSGISTYYTDSVKGRFTTSRDNPKNTLFLQMSSLRAEDTAVYYCAKGIVGATTPFDYWGQGTLVTVSS

SEQ ID NO: 39 - protein, 108 aa, synthetic construct
EIVLTQSPATLSLSPGERATLSCRASQSVRSFLAWYQQKPGQAPRLLIYETSNRATGIPARFSGSGSGTDFTLTISSLEPEDFAVYYCQQRSDWPPTFGQGTKVEIKR

SEQ ID NO: 40 - protein, 119 aa, synthetic construct
QVQLVQSGPGLVKPSETLSLTCTVSGGSISYYYWSWIRQPPGKGLEWIGHISYSGSTNYNPSLKSRVTISEDTSKKQFSLKLDSVTAADTAVYYCAGGIVGARRGFDFWGQGTLVTVSS

SEQ ID NO: 41 - protein, 108 aa, synthetic construct
EIVMTQSPATLSLSPGERATLSCRASQSVSNNLAWYQQKPGQAPRLLIYDASNRAADIPARFSGSGSGTDFTLTISSLEPEDFAVYYCQQRNNWPPTFGQGTKVEIKR

SEQ ID NO: 42 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 43 - protein, 121 aa, synthetic construct
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 44 - protein, 107 aa, synthetic construct
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKR

SEQ ID NO: 45 - protein, 120 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 46 - protein, 113 aa, synthetic construct
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRR

SEQ ID NO: 47 - protein, 116 aa, synthetic construct
EVQLVESGGGLVKPGGSLRLSCAASGFTFSDYGMNWVRQAPGKGLEWIAYISSGRGNIYYADTVKGRFTISRDNAKNSLYLQMNSLRAEDTAVYYCARSWGYFDVWGQGTTVTVSS

SEQ ID NO: 48 - protein, 114 aa, synthetic construct
DIVMTQSPDSLAVSLGERATINCKSSQSLLNRGNQKNYLTWFQQKPGQPPKLLIYWASTRESGVPDRFSGSGSGTDFTLTISSLQAEDVAVYYCQNDYTYPLTFGQGTKLEIKR

SEQ ID NO: 49 - protein, 289 aa, synthetic construct
MVSLPRLCALWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 50 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 51 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 52 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 106 aa, synthetic construct; identical to SEQ ID NO: 60

SEQ ID NO: 63 - protein, 330 aa, synthetic construct; identical to SEQ ID NO: 54

SEQ ID NO: 64 - protein, 106 aa, synthetic construct; identical to SEQ ID NO: 60

SEQ ID NO: 65 - protein, 124 aa, synthetic construct
EVQLVQSGGGVVQPGRSLRLSCAASGFSFSSYAMHWVRQVPGKGLEWVSVISYDGDNKYHAESVKGRFTISRDNSKNILYLQMNSLRPEDTAVYYCARRGTVTNYYSYSGLDVWGQGTTVTVSS

SEQ ID NO: 66 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 121 aa, synthetic construct [sequence omitted]

SEQ ID NO: 68 - protein, 107 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR